(19) United States
(12) Patent Application Publication
(10) Pub. No.: US 2015/0013024 A1
(43) Pub. Date: Jan. 8, 2015

(54) NEAR-INFRARED LIGHT-ACTIVATED PROTEINS

(71) Applicant: University of Wyoming, Laramie, WY (US)

(72) Inventors: Mark Gomelsky, Laramie, WY (US); Min-Hyung Ryu, Laramie, WY (US)

(73) Assignee: UNIVERSITY OF WYOMING, Laramie, WY (US)

(21) Appl. No.: 14/326,778

(22) Filed: Jul. 9, 2014

Related U.S. Application Data
(63) Continuation-in-part of application No. 13/560,645, filed on Jul. 27, 2012, now Pat. No. 8,835,399.
(60) Provisional application No. 61/512,065, filed on Jul. 27, 2011.

(51) Int. Cl. C12N 9/88; C07K 7/06; C12N 9/50

(52) U.S. Cl. CPC C12N 9/88; C12N 9/50; C07K 7/06; C12Y 406/01001; C12Y 304/22056; C07K 2319/50
USPC 800/13; 435/232; 435/212; 536/23.4; 435/252.33; 435/471; 514/44 R

(57) ABSTRACT

Methods and constructs are provided for controlling processes in live animals, plants or microbes via genetically engineered near-infrared light-activated or light-inactivated proteins including chimeras including the photosensory modules of bacteriohytochromes and output modules that possess enzymatic activity and/or ability to bind to DNA, RNA, protein, or small molecules. DNA encoding these proteins are introduced as genes into live animals, plants or microbes, where their activities can be turned on by near-infrared light, controlled by the intensity of light, and turned off by near-infrared light of a different wavelength than the activating light. These proteins can regulate diverse cellular processes with high spatial and temporal precision, in a nontoxic manner, often using external light sources. For example, near-infrared light-activated proteins possessing nucleotidyl cyclase, protein kinase, protease, DNA-binding and RNA-binding activities are useful to control signal transduction, cell apoptosis, proliferation, adhesion, differentiation and other cell processes.

### CROSS-REFERENCE TO RELATED APPLICATIONS

This application claims priority to U.S. Provisional Patent Application No. 61512065 filed Jul. 27, 2011, and is a continuation-in-part of U.S. patent application Ser. No. 13/560,645 filed Jul. 27, 2012, both of which are incorporated herein by reference to the extent not inconsistent herewith for purposes of written description and enablement.

### STATEMENT REGARDING FEDERALLY SPONSORED RESEARCH

This invention was made with Government support under NIH Contracts No. 2P20 RROI6474-09 and R21 CA167862. The Government has certain rights in the invention.

### BACKGROUND

Light-activated fluorescent proteins have revolutionized imaging technologies, and with them our fundamental understanding of cellular processes (Zimmer, 2009). The use of light to control protein activities in live animals with spatiotemporal resolution unmatched by drugs has even greater potential (Miesenböck et al. 2009; Liu & Tonegawa, 2010). Optogenetic approaches utilizing natural photoreceptors have provided insights into the underpinnings of information processing in the nervous system, locomotion, awakening, neural circuits in Parkinson's disease, progression of epilepsy, etc. (Airan et al., 2009; Adamantidis et al., 2007; Cardin et al., 2009; Gradinaru et al., 2009; Sohal et al., 2009; Tønnesen et al., 2009; Tsai et al., 2009; and Gradinaru et al., 2010). Several groups have succeeded in engineering photoactivated proteins with new functions (Lee et al., 2008; Strickland et al., 2008; Tyszkiewicz and Muir, 2008; Yazawa et al., 2009; Möglich et al., 2009; Wu et al., 2009; and Georgianna & Deiters, 2010). However, the use of optogenetic approaches outside neurobiology remains very limited (reviewed in Möglich et al., 2010; Toettcher et al., 2011). The potential of using proteins activated by far-red and near-infrared (NIR) light, which penetrates animal tissues to the depths of several centimeters (Cuberddu et al., 1999; Wan et al., 1981; Byrnes et al., 2005) and therefore can be applied externally, has remained largely unexplored because of limitations in the ability to engineer such proteins with desired output activities.

### SUMMARY

The ability to precisely activate or inactivate desired proteins in vivo—in specific cells or tissues of live animals, during normal or disease conditions—offers unprecedented insights into understanding diverse biological processes. However, current genetic and pharmaceutical approaches do not provide the spatiotemporal resolution and/or target specificity to accurately interrogate cellular functions in real time in vivo.

Light has emerged as an alternative means to control cellular activities with spatiotemporal precision unattainable by other approaches. The recently emerged field of optogenetics involves delivery into model organisms of recombinant genes encoding proteins that can be turned “on” and “off” by light. While natural photoactivated proteins (e.g., channelrhodopsins) have revolutionized neurobiology, the enormous potential of engineered photoactivated proteins remains largely untapped. We have elucidated principles of engineering far-red/NIR light-activated proteins using photosensory modules of bacteriophytochromes, a subclass of phytochromes containing the biliverdin IXα chromophore (Rockwell & Lagarias, 2006). Far-red/NIR light penetrates animal tissues much deeper (centimeter scale) than visible light (millimeter scale) absorbed by currently used photoreceptors; therefore bacteriophytochromes are particularly attractive and potentially transformative for optogenetic applications in mammalian models of development and disease as well as for disease treatment.

Applicants have designed bacteriophytochrome-based homodimeric photoactivated proteins and provide principles for engineering a broad spectrum of photoactivated functions. A large fraction of important signal transduction proteins operate as homodimers, e.g., membrane receptors, protein kinases, protein phosphatases, proteases, nucleases, and transcription factors. Three-dimensional structures of many of these proteins are known to the art. All these proteins represent targets for protein engineering.

“Transplantation” of a phytochrome photoreceptor module has been achieved previously, albeit only to homologous downstream domains (Levskaya et al., 2005, 2009). Phytochromes have also been designed to control protein-protein interactions in a light-dependent manner (Leung et al., 2008). However, the present disclosure is the first to provide photosensory modules of bacteriophytochromes to directly activate heterologous outputs. No such engineered modules have previously been available, and specifically, no light-activated bacteriophytochrome-based nucleotidyl cyclases or caspases have previously been available.

Provided herein are methods of controlling processes in live animal, plant or microbial organisms via genetically engineered far-red/NIR-light activated homodimeric proteins, NIRLAHPs. These proteins are chimeras comprised of photosensory modules of bacteriophytochromes that are activated (or inactivated) by far-red/NIR light and output modules that possess enzymatic activity and/or ability to bind to DNA, RNA, protein, or small molecules.

In this application, the term “NIR light” is used to describe light of 700-3000 nm wavelengths, commonly defined as NIR or infra-red A (IR-A), as well as an adjacent region of far-red light of 650-700 nm wavelengths.

Genes encoding NIRLAHPs can be introduced into live animals, plants or microbes, where their activities can be turned on by NIR light, controlled by the duration and/or intensity of light, and turned off by light of a different wavelength than the activating light. By using NIRLAHPs one can regulate diverse cellular processes with high spatial and temporal precision in a nontoxic manner, often using external light sources. For example, NIRLAHPs possessing nucleotidyl cyclase, protein kinase, protease, DNA-binding and RNA-binding activities can be used to control metabolic enzymes, signal transduction, cell apoptosis, proliferation, adhesion, differentiation and other processes. These features of NIRLAHPs can be used in various medical applications. For example, a NIR light-activated executor (effector) caspase can be introduced into tumors (or other kinds of disease-causing cells, e.g., cells carrying viruses) to induce an apoptotic cell death pathway, thus providing a noninvasive gene therapy of cancer (or viral diseases). Human cells expressing hormones (e.g., insulin) can be regulated by NIRLAHPs (e.g., due to the light-regulated gene expression or hormone-synthesizing activity) and can be used to treat hormone deficiencies (e.g., diabetes). NIRLAHPs can be used to photoactivate immune cells at desired locations (e.g., tumor or infection sites). NIRLAHPs can also be used to convert prodrugs into active drugs in irradiated tissues and/or organs. NIRLAHPs expressed in bacteria (e.g., E. coli or Lactobacillus) that belong to normal human or animal microflora can be used to photoactivate organ-localized (e.g., colon, vagina) synthesis of bacteriophages, antibiotics, and other drugs to target pathogenic microorganisms, polyps and tumors or to produce probiotics. Some NIRLAHPs can be used as protein-based drugs directly (e.g., by light-activated binding and control of cellular receptors). NIRLAHPs can also be used in cell-based nanomanufacturing (by virtue of light-dependent cell growth or light-dependent production of a desired product), and in industrial applications (e.g., light-induced dissolution of bacterial biofilms formed in the presence of engineered near-infrared light-sensitive cells that secrete biofilm-dispersion agents).

The principal advantages of NIR light over ultraviolet (UV) and visible light, which are sensed by all other types of photoreceptor proteins, is superior penetration into biological tissues (centimeter scale) and lack of toxicity. Therefore, activities of NIRLAHPs can be controlled in tissues that are not accessible to UV and visible light (e.g., most animal tissues); they can be controlled not only by implanted light sources, but in many cases, by external light sources (e.g., by lasers or light-emitting diodes, LEDs, placed outside organisms that are being controlled). Additional advantages of bacteriophytochrome-based NIRLAHPs involve their capacity for instant photoinactivation (usually by light of a longer wavelength than the activating light); lack of known toxicity of NIR light; and lack of toxicity, at low doses, of the chromophore biliverdin IXα, which is naturally present in most animal tissues or can be supplied via injection, diet, or via synthesis in vivo by a heterologous heme oxygenase gene.

Methods are provided herein for producing photoactive fusion proteins having a desired activity controllable by NIR light, said method comprising the steps: a. designing one or more homodimeric fusion proteins, each comprising a photoreceptor protein module and a heterologous output module, wherein: i. said homodimeric fusion proteins comprise two monomers that each comprise: (1) a photoreceptor module of a bacteriophytochrome; and (2) a heterologous output module capable of being activated upon homodimerization to perform said desired activity; and ii. said monomers are not active when separated, but are capable of combining to form homodimers that are controllable by NIR light; wherein designing said fusion proteins comprises identifying candidate output domains based on 3D structures or structural models, identifying candidate protein fusion sites and estimating lengths of α-helices linking said output modules to said photosensory modules; b. producing a plurality of DNA molecules, each encoding a said monomer of a said homodimeric fusion protein that has at least one unique fusion site; c. screening said DNA molecules for their ability to produce homodimeric photoactive fusion proteins capable of performing said desired activity by a method comprising: transforming a designed test organism with a plurality of different said DNA molecules such that different said fusion proteins are expressed in each test organism; ii. allowing the expressed fusion proteins to bind bacteriophytochrome chromophore and form homodimeric proteins; and iii. applying selected wavelengths of NIR light to said transformed organisms and determining the level of said desired activity of said fusion proteins in said organisms in the presence and absence of said selected wavelengths of light; wherein the level of said desired activity of said fusion proteins is controllable by NIR light when the level of said desired activity is changed by the presence and/or absence of NIR light having said selected wavelengths. Controllability by NIR light of the fusion proteins exists when the fusion proteins have higher ratios of activity in the light versus dark or vice versa.

The bacteriophytochrome photoreceptor module can be from the BphG1 protein from Rhodobacter sphaeroides. The test organism for expression of said fusion protein can be a cultured organism selected from the group consisting of E. coli, yeast, plant, and animal cells selected or modified so as to detectably exhibit the level of activity of said expressed fusion protein controllable by the presence or absence of NIR light. Examples of light-activated fusion proteins produced by the methods hereof are light-responsive nucleotidyl cyclases and light-responsive uncleavable procaspase-3.

The test organisms can comprise an endogenous chromophore or they may not. If required, they are transformed with DNA encoding a heme oxygenase gene capable of being expressed therein to produce a biliverdin Ixα chromophore, e.g. the BphO1 protein from Rhodobacter sphaeroides.

The method also comprises modifying the design of the fusion proteins that are controllable by NIR light to produce additional candidate fusion proteins by designing additional fusion sites and linkers for said fusion proteins and repeating the steps of producing DNA encoding the additional fusion proteins, transforming suitable organisms with this DNA, expressing the DNA, and screening the resultant fusion proteins for additional fusion proteins controllable by NIR light. This is achieved by increasing or decreasing the lengths and amino acid sequences of the α-helical linkers linking the photoreceptor modules with the output modules, e.g., the linker lengths can be increased or decreased by three or four amino acids, representing one full turn of the linker strand.

Fusion proteins controllable by NIR light, or additional fusion proteins controllable by NIR light produced by increasing or decreasing their linker lengths, can be mutagenized to create further candidate fusion proteins controllable by NIR light, followed by repeating the screening steps to identify photoactivated fusion proteins with improved properties, e.g. low background activity and high photoactivation ratio.

In various embodiments, fusion proteins are produced by the methods hereof whose activity can be increased by the application of NIR light of a selected wavelength, or can be decreased by the application of NIR light of a selected wavelength. In embodiments, the desired activity can be gradually decreased or gradually increased by ceasing to apply NIR light of a selected wavelength or by application of NIR light of a selected wavelength.

Provided herein are homodimeric fusion proteins controllable by NIR light, said fusion proteins comprising a photoreceptor module comprising a bacteriophytochrome and a heterologous output module capable of producing a desired activity, e.g., light-activated nucleotidyl cyclases or light-activated uncleavable procaspase-3. Recombinant DNA molecules encoding the homodimeric fusion proteins hereof are also provided.

In addition, methods are provided herein for controlling an in vivo process in a host, which is a living cell or organism using the fusion proteins hereof. The method comprises: a. introducing into the cell or organism a DNA sequence encoding a homodimeric fusion protein comprising a photoreceptor module comprising a bacteriophytochrome and a heterologous output module capable of modulating said process; b. allowing said fusion protein to be expressed in said host; and c. applying NIR light of a selected wavelength to the host or preventing NIR light of a selected wavelength from reaching the host; thereby modulating the process under control of NIR light. Such processes can be selected from the group consisting of metabolic processes, signal transduction, cell apoptosis, cell proliferation, cell adhesion, and cell differentiation.

In embodiments hereof, methods hereof for producing NIRLAHPs having a desired activity controllable by NIR light comprise the steps of designing one or more homodimeric fusion proteins, each comprising a bacteriophytochrome photoreceptor module and a heterologous output module, capable of being NIR-light activated to perform said desired activity. The monomers of the fusion proteins combine spontaneously to form homodimers, and have autocatalytic activity to bind biliverdin IXα, thus forming NIRLAHPs. Designing NIRLAHPs comprises (a) identifying, based on biochemical information candidate protein output domains that function as homodimers and can be activated by homodimerization; (b) using 3D structures or building 3D models to identify optimal fusion sites and peptide linkers for attaching the heterologous output modules to the bacteriophytochrome photoreceptor modules; (c) producing a plurality of DNA molecules (a DNA library), each encoding a monomer of a homodimeric fusion protein that has at least one unique fusion site or linker sequence; and (d) screening the DNA molecules for their ability to produce homodimeric photoactive fusion proteins capable of performing the desired activity in a test organism. The screening is done by transforming a test organism designed to respond to the desired activity with the DNA constructs encoding the monomers of the homodimeric fusion proteins; allowing the expressed fusion proteins to spontaneously bind biliverdin IXα and form homodimers; applying light of selected wavelengths to the transformed organisms; and comparing the level of said desired activity of the expressed fusion proteins in the test organism in the dark and in the light. The method can then comprise (e) subjecting fusion proteins that have NIR light-activated activities identified by screening to random mutagenesis and subsequent screening (by the method described above) for mutant derivatives with improved qualities, e.g., low activity in the dark and a high light-to-dark activation ratio. The method can further comprise: (f) purification, and spectral and/or biochemical characterization of NIRLAHPs.

The optimized NIRLAHPs can be used for controlling in vivo processes in other organisms, including animals and humans, using internal and/or external sources of NIR light. The genes encoding NIRLAHPs can be introduced into the cell or organism via methods known to the art, including transformation by DNA, viral infection, and bacteriofection.

Light-activated fusion proteins, DNA molecules encoding them, and methods for using them to control processes in living hosts are also provided herein.

### BRIEF DESCRIPTION OF THE DRAWINGS

The following drawings illustrate various aspects of the photoreceptor modules for optogenetic applications provided in the present disclosure.

FIG. 1 shows six major photoreceptor types (Gomelsky & Hoff, 2011) (top panel) including a subclass of phytochromes known as bacteriophytochromes. The photoreceptor module from the BphG1 protein from Rhodobacter sphaeroides (Tarutina et al., 2006) was used into illustrate the present methods. The molecular structure of the chromophore of the BphG1 protein (biliverdin IXα) is shown in the lower right panel. The lower left panel shows light-induced spectral changes in the BphG1 protein. The protein exists in the “dark” (Pr) form (absorption maximum 712 nm) when it is not exposed to light or irradiated with light of ˜740-780 nm. Upon irradiation with light of ˜650-715 nm, the protein is converted to the “lit” (Pfr) form (absorption maximum ˜760 nm).

FIG. 2 (image from Cubeddu et al., 1999) illustrates major advantages of biliverdin IXα containing bacteriophytochromes as photoreceptor modules for engineering light-activated proteins for use in mammals. These advantages include deep penetration of NIR into mammalian tissue, lack of toxicity, ubiquity of biliverdin IXα in mammalian tissue (as natural product of heme turnover) and instant photoinactivation. Left top panel: absorbance of light passing through flesh of a human hand. Right top panel: the absorbance of human breast tissue at different wavelengths. Arrows approximately delimit the range of the spectrum with low light absorption by human tissues, which provide for deeper light penetration.

FIG. 3 (image from Weissleder, 2001) shows the “NIR window” from about 670 nm to about 890 nm, where cumulative absorption by three major light-absorbing components of flesh in red-blooded animals, deoxyhemoglobin (Hb), oxyhemoglobin (HbO₂) and water (H₂O), is lowest. The NIR window identifies the range of wavelengths that can be used for deepest penetration through mammalian tissues. Bacteriophytochrome absorption peaks fall into the NIR window.

FIG. 4 illustrates protein domain architecture (top panel) as well as spectral and enzymatic properties (bottom panel) of the BphG protein used for protein engineering. Top panel, R. sphaeroides BphG (PAS-GAF-PHY-GGDEF), a derivative of BphG1 (PAS-GAF-PHY-GGDEF-EAL) lacking the EAL domain (Tarutina et al., 2006). BphG converts two guanosine triphosphate (GTP) molecules into cyclic dimeric GMP (c-di-GMP) by means of a diguanylate cyclase activity of the GGDEF domain (Ryjenkov et al., 2005). Bottom panel, left: spectral properties of BphG (same as in FIG. 2). Bottom panel, right: synthesis of c-di-GMP by R. sphaeroides BphG in vitro, in the dark (grey line) and in the light (black line). BphG has an approximately 10-fold photoactivation ratio (relative activity in the light divided by relative activity in the dark).

FIG. 5A is a schematic depiction of the R. sphaeroides BphG protein comprising the photoreceptor (PAS-GAF-PHY) and output (GGDEF) modules. The BphG protein is depicted as a parallel homodimer. FIG. 5B is a 3D model of the BphG protein based on the 3D structure 3c2w for the phototreceptor module and 3icl for the output domain (3D structures from Protein Data Bank [PDB], rcsb.org/pdb). The dashed line represents the approximate position within the α-helices (extending from the photoreceptor PHY domain) for fusion with a heterologous homodimeric output module. An arrow indicates rotation of an output domain as a potential outcome of light-induced conformational changes in BphG. FIG. 5C depicts a 3D model of the homodimeric adenylyl cyclase domains of protein CyaB1 from Nostoc sp. (modeled based upon the PDB structure 1wc5, the protein with the highest sequence identity to CyaB1). The output module of R. sphaeroides BphG, the diguanylyl cyclase GGDEF domain, was replaced with a distantly related adenylyl cyclase (ACyc) domain from Nostoc sp. CyaB1 resulting in the photoactivated adenylyl cyclase. FIG. 5D is a schematic representation of the protein domain architecture (GAF-PAS-ACyc) of Nostoc sp. CyaB1 depicted as a homodimer.

FIG. 6 illustrates the use of E. coli as a test organism to screen a DNA library of fusion protein constructs for adenylyl cyclase activity. A library of fusion proteins encoded by the “chimeric AC (adenylyl cyclase) genes” is expressed (e.g., from a P_{BAD} promoter) in the E. coli strain having a cya gene deletion, which does not naturally produce cAMP. If the fusion protein generates cAMP, it enables expression of the (CRP-cAMP)-dependent lacZYA operon, which results in blue colonies on XGal indicator plates. In the absence of cAMP, the colonies are colorless. By comparing the color of colonies grown in the light (650-nm LED panel) and in the dark, one can identify light-activated and light-inactivated versions of adenylyl cyclases. The colonies appearing blue in the light and colorless (or less blue colored) in the dark contain candidate light-activated adenylyl cyclases. The colonies appearing colorless in the light and blue in the dark contain candidate light-inactivated adenylyl cyclases.

FIG. 7 illustrates XGal indicator plates containing representative E. coli clones, each having a gene encoding a different (numbered) fusion adenylyl cyclase protein construct, grown either in the dark (left panel) or in the light (right panel). Blue color indicates how each clone responds to light. The white arrowhead points to the clone containing a light-activated adenylyl cyclase having the sequence: MAQRTRAELERKEVT [SEQ ID NO:6] the black arrowhead points to one of the light-inactivated adenylyl cyclases, RlaC18, having the sequence MAQRTRAELARLRHYDERKEVT [SEQ ID NO:1].

FIG. 8 shows images of E. coli clones containing selected light-activated RlaC17 (SEQ ID NO:6), RlaC25, MAQRTERKEV T [SEQ ID NO:10] and light-inactivated RlaC18, (SEQ ID NO:1) fusion adenylyl cyclase proteins grown in the dark and light, along with adenylyl cyclase activities of the purified proteins measured under light and dark conditions in vitro.

FIG. 9 provides protein sequences near fusion points of selected engineered adenylyl cyclase fusions between the photoreceptor module of BphG and adenylyl cyclase (ACyc domain) of CyaB1. Photoresponses in E. coli of the fusion proteins were recorded at two levels of expression of the chimeric proteins: low (5 μM isopropyl-beta-D-thiogalactopyranoside, IPTG) and high (50 μM IPTG). 3-galactosidase expression (judged by the intensity of blue color) is dependent on intracellular cAMP levels. A: light-activated (higher β-galactosidase expression in the light versus dark); I: light-inactivated (higher β-galactosidase expression in the dark versus light); +: light-independent activity; −: no activity (in the dark or light). It is emphasized that protein fusions containing approximately one helical turn (+/−3-4 amino acids, aa) longer or shorter α-helical sequences had the same type of light-responsiveness, e.g., light-activated RlaC17 (SEQ ID NO:6); RlaC29 MAQRTRAELARLRERKEVT [SEQ ID NO:2]; RlaC17 (SEQ ID NO:6+4 aa); RlaC25 (SEQ ID NO:10) (which has the sequence of SEQ ID NO:6−4 aa); and RlaC22 MAQRTRERKEVT [SEQ ID NO:9](which has the sequence of SEQ ID NO:6−3 aa).

With respect to the remaining constructs shown in FIG. 9, RlaC28 whose sequence is MAQRTRAELARLERKEVT [SEQ ID NO: 3] showed activity. RlaC24, whose sequence is MAQRTRAELARERKEVT [SEQ ID NO: 4] showed no activity, as did RlaC23, whose sequence is MAQRTRAELAERKEVT [SEQ ID NO:5], RlaC30, whose sequence is MAQRERKEVT [SEQ ID NO:11], RlaC26, whose sequence is MAQERKEVT [SEQ ID NO:12] and Rlac27, whose sequence is MAERKEVT [SEQ ID NO:13].

FIG. 10 illustrates an overview of principles elucidated herein. The photosensory module of a bacteriophytochrome is shown schematically in the lower left with a modeled heterologous active module shown above. In the center of the figure, a modeled photosensory module is shown with the active module symbolized by a “homodimeric head”. On the right, a generic modeled active module is illustrated above a list of examples of modules that can be used to engineer NIRLAHPs, e.g., kinases, proteases, transcription factors, nucleases, etc.

FIG. 11 illustrates domain architectures, and 3D models of the components used in infrared light-activated adenyl cyclase (IlaC) engineering. R. sphaeroides bacteriophytochrome BphG is bacteriophytochrome DGC; Nostoc sp CyaB1 is homodimeric adenylyl cyclase. In this application, RlaC and IlaC are used interchangeably.

FIG. 12 illustrates IlaC engineering strategy, a subset of BphG-CyaB1 protein fusion sequences, their AC activities and responsiveness to light. The aa shades of gray correspond to the shades of protein domains shown above protein sequences. Interdomain linkers are shown in black. Predicted secondary structure elements, α-helix and β-strand, are shown above sequences. AC activity: +, active; −, inactive, according to the lacZ plate assays (see panel B). Response to light: −, no response; ↑, activation; ↓, inactivation.

FIG. 13 shows images of the lacZ plate assays of AC activity. Selected E. coli BL21 [DE3](pETilaC#; pT7-ho1-1) strains from panel A were grown on LB agar containing X-gal and IPTG, either in the dark (left of center) or in the red light (right). Blue colony color indicates cAMP-CRP-induced lacZ expression. Each strain expressing an IlaC# was plated in a sector of each of the 4 plates. The plating guide is in the center of the panel. IlaC expression from pETilaC# was induced at two IPTG levels: 10 μM (top plates) and 50 μM (bottom plates).

FIG. 14 shows kinetics of cAMP accumulation by the purified IlaC proteins in the dark and light. Top row, first-generation light-responsive proteins: IlaC18, light-inactivated AC; IlaC17, 22 and 25, light-activated AC. Bottom row, IlaC22 mutants with improved photodynamic ranges: IlaC22 R509W and IlaC k27. AC activity was measured at room temperature. cAMP was quantified by high pressure liquid chromatography (HPLC). Black traces, dark (dim green light); gray traces, irradiation with 700-nm light.

FIG. 15 shows photochemical characterization of IlaC variants. (A) Kinetics of the dark recovery of IlaC derivatives from the lit (Pfr) state. Plotted are changes in absorbance at 755 nm over time following 5-min excitation with 700-nm light. Bottom trace, IlaC22 k27; top trace, IlaC22 k27 Y259F. Half of the Pfr form of IlaC22 k27 decayed after 46±3 s and half of the Pfr form of the IlaC22 k27 Y259F mutant decayed after 197±9 s. (B) Plate assays of the IlaC k22 dark recovery mutants. (C) Light-induced spectral changes in IlaC22 derivatives. Top trace (Pr), prior to irradiation, middle trace (Pfr), after irradiation with 700-nm light; Δ, difference (Pfr−Pr) spectra. IlaC22 k27, left panel; IlaC22 k27 Y259F, right panel.

FIG. 16 shows red light-stimulated locomotion of animals expressing IlaC22 k27 in C. elegans cholinergic neurons (Punc-17:ilaC). (A) When exposed to environmental light on an agar surface, transgenic Punc-17:IlaC animals have higher frequency of anterior body bends relative to wild-type animals (two-tailed Student's test, **p<0.001, N≧12). (B) Red (650 nm) light increases the number of body bends performed on an agar surface by Punc-17:IlaC animals (two-tailed Wilcoxon Rank-Sum test, §p<0.05, N=11). (C) Red light increases the thrashing rate in liquid of Punc-17:IlaC animals (two-tailed Wilcoxon Rank-Sum test, §p<0.05). After re-exposure to green light (532 nm; 61-120 s interval), Punc-17:IlaC animals thrash slower compared to their first green light exposure (two-tailed Wilcoxon Rank-Sum test, §§p<0.005) and compared to wild-type controls (two-tailed Student's t-test, *p<0.05, Na≧20). Two transgenic strains were analyzed, both showing similar results. Analysis of body bends on an agar surface was performed on the strain NQ721 and analysis of the thrashing rate in liquid was performed on the strain NQ719. All experiments with C. elegans were performed by Mathew D. Nelson and David M. Raizen (University of Pennsylvania).

FIG. 17 shows structural models of BphG and the AC domain of CyaB1. The parallel dimer of the phytochrome domains was constructed using two PDB structures, combined in several steps.

FIG. 18 shows a selection strategy used for isolating IlaC22 R509W mutants with decreased dark AC activities.

FIG. 19 illustrates identification of residues involved in slowing the dark thermal recovery of IlaC. Shown is alignment of the R. sphaeroides BphG bacteriophytochrome and the Arabidopsis thaliana phytochrome PhyB. Residues of PhyB affecting its photocycle and conserved in BphG are highlighted in dark gray Mutations of PhyB that extend the lit (Pfr) state of PhyB are shown in light gray.

FIG. 20 shows effects of IlaC k27 and IlaC k27 Y259F on cAMP-CRP-dependent lacZ gene expression in E. coli. Strain BL21[DE3] cya (pT7-ho1-1) containing either pET-ilaCK27 or pET-ilaC k27 Y259F were grown in culture tubes on a shaking platform at 30° C. in LB supplemented with ampicillin (50 μg/mL) and kanamycin (50 μg/mL).

All publications and websites disclosed herein are incorporated by reference to the extent not inconsistent herewith.

### DETAILED DESCRIPTION

### Definitions

Terms used herein have their generally accepted, conventional meaning in the art unless otherwise specifically defined.

A “fusion protein” hereof (also referred to herein as an “engineered protein,” a “chimeric protein” and/or a “hybrid protein”) is a protein that comprises an output module and a photosensory module that do not occur together in the same protein in nature.

An “output module” (also referred to herein as an “output domain”) is the portion of a protein that performs a function, e.g., enzymatic activity, or binding to DNA, RNA or another protein.

A “photosensory module” (also referred to herein as a “photoreceptor module”) is a portion of a protein that contains a chromophore, through which it senses and responds to light.

A “chromophore” is a molecule bound to the photoreceptor module that serves to detect NIR light and cause a conformational change in the output domain of the fusion protein when NIR light is applied. In bacteriophytochromes, the chromophore is biliverdin IXα.

A homodimer is a protein having two identical portions (monomers) that are not linked to each other by covalent bonds but can form stable structures involving protein-protein (monomer-monomer) interactions. In the homodimeric fusion proteins hereof that are photoactive, monomers making up the homodimeric proteins are inactive until they have joined to form a particular homodimeric conformation.

Bacteriophytochromes are a subclass of phytochrome photoreceptor proteins containing biliverdin IXα as a chromophore. The photosensory modules of biliverdin IXα comprise PAS-GAF-PHY protein domains. Bacteriophytochromes covalently bind biliverdin IXα to a conserved cysteine residue via an intrinsic biliverdin ligase activity.

“Near infrared” (NIR) light is generally considered in the art to have a wavelength of between about 700-750 and about 3000 nm. “Far-red” light is generally defined as light having a wavelength at the long-wavelength red end of the visible (red) spectrum, from about 700 to about 750 nm. The visible spectrum is generally defined as having a wavelength of about 390 to about 750 nm. Bacteriophytochromes sense light from about 650 to about 800 nm, within the “NIR window.” Since this “NIR window” contains light variously defined as being in the visible, far-red and NIR categories, the term “near-infrared” (“NIR”) is used herein to describe light in the “NIR window” that activates bacteriophytochromes, switching them from one (dark) conformation to another (lit) conformation and back, regardless of whether the light would be generally defined as being in the NIR range, the far-red range, or in the visible range.

The term “light activation” (also referred to herein as “photoactivation”) is used herein to refer to control of a protein activity by application of NIR light of selected wavelengths or removal of light from a fusion protein as described herein. The fusion protein is “activated” when NIR light applied to the photoreceptor causes a change in conformation of the output module of the fusion protein such that it changes the activity of the output module. This change is believed to be caused, at least in part, by rotation of the monomeric output modules with respect to each other such that a desired activity of the fusion protein is changed, e.g., stopped, started, enhanced, or decreased. The term “light activated” (also called “photoactive” in reference to proteins hereof) means a protein capable of being controlled by NIR light to be active or inactive, or more or less active or inactive. Thus, the terms “photoactive proteins” or “photoactivated proteins” also include “photoinactive proteins” or “photoinactivated proteins,” respectively.

A “photoactivation ratio” (also referred to as a “light-activation ratio” or “dynamic range”) is the ratio of protein activity upon NIR irradiation to protein activity in the dark. In embodiments, the protein activity can be achieved by applying light of selected wavelength to the protein, or by removal of such light. In embodiments, the protein can be made active by applying light of a selected wavelength and can be made immediately inactive by applying light of a different selected wavelength, or can be allowed to become gradually inactive by removing light of said different selected wavelength. In embodiments, the protein can be made inactive by applying light of a selected wavelength and can be made immediately active by applying light of a different selected wavelength, or can be allowed to become gradually active by removing said light of a different selected wavelength. In embodiments, the fusion proteins hereof can be controlled to be substantially completely inactive or substantially completely inactive by the foregoing means (when high light activation ratios are achieved), or can be controlled to be relatively inactive or to be relative active (when low light activation ratios are achieved).

A “fusion site” defines the amino acid of the photoreceptor module that is linked to the specific amino acid of the output module of the fusion protein.

A “linker region” of fusion protein hereof is the α-helical protein region that includes a fusion site. The linker region of the fusion protein may be composed entirely of α-helical regions or partly of α-helical region. Linker regions hereof may be shortened or lengthened using amino acid sequence of the photoreceptor module or artificial sequence in order to cause or improve control of activity of NIRLAHPs by light.

A “plurality” as used herein means two or more.

Microbial photoreceptors, bacteriophytochromes, absorb near-infrared light, which penetrates deep into animal tissues and is harmless. Bacteriophytochromes delivered as genes can be used to control biological activities in live animals via external light sources. However, the lack of understanding of light-induced conformational changes has hindered development of bacteriophytochrome-based optogenetic tools. Here, we show that homodimeric bacteriophytochromes can be engineered to activate heterologous output domains that require homodimerization.

Light has advantages over chemical means of regulating biological processes because it acts noninvasively and provides superior spatial and temporal resolution. Optogenetic approaches that rely on algal and archael channel rhodopsins activating specific animal neurons opened up a new era in neurobiology. Photoreceptors of several other types have been engineered to regulate biological processes and used in cell cultures and transparent animals (Pathak et al., 2013; Müller and Weber, 2013). However, application of optogenetic tools in heme-rich animal tissues has been hindered by high scattering and poor penetration of visible light. Light in the near-infrared window (NIRW), which encompasses the spectral region of approximately 680-880 nm, penetrates animal tissues much better than light outside NIRW (Weissleder, 2001). A significant fraction of NIRW light can pass through several cm of human tissues (Wan et al., 1981; Cubeddu et al., 1999; Byrnes et al., 2005), which makes it possible to control biological processes in animals using NIRW light. Absence of photoreceptors of NIRW light in most animal tissues is an additional advantage that makes NIRW light harmless (Piatkevich et al. 2013). This is in contrast to blue light, which is absorbed by flavins and porphyrins, and therefore promotes photooxidative damage (Hockberger et al., 1991).

Phytochromes are photoreceptors that absorb light in the NIRW of the spectrum (Rockwell et al. 2006; Auldridge and Forest 2011; Ulijasz and Vierstra 2011). The photosensory modules of these photoreceptors covalently bind bilin chromophores. Plant and cyanobacterial phytochromes bind phycocyanobilins or phycoerythrobilins, while bacteriophytochromes bind biliverdin IX.

As the first product of heme turnover, biliverdin IXα is naturally present in animal cells, which makes bacteriophytochromes preferred over plant and cyanobacterial phytochromes, whose chromophore synthesis requires dedicated enzymes. Further, absorption wavelength maxima of bacteriophytochromes are red-shifted compared to the absorption maxima of plant and cyanobacterial phytochromes. This results in a 2-10-fold gain in the penetration depth of light through mammalian tissues (Wan et al., 1981; Piatkevich et al., 2013). Up to now, bacteriophytochrome engineering for optogenetic applications has lagged behind the engineering of photoreceptors of other types (Pathak et al., 2013), including engineering of plant phytochromes (Levskaya et al., 2009).

The major obstacle to bacteriophytochrome engineering has been the lack of understanding of the mechanisms though which light induced conformational changes are transduced to regulate output activities (Rockwell et al., 2006; Auldridge and Forest, 2013; Ulijasz et al., 2011; Mdglich et al., 2010).

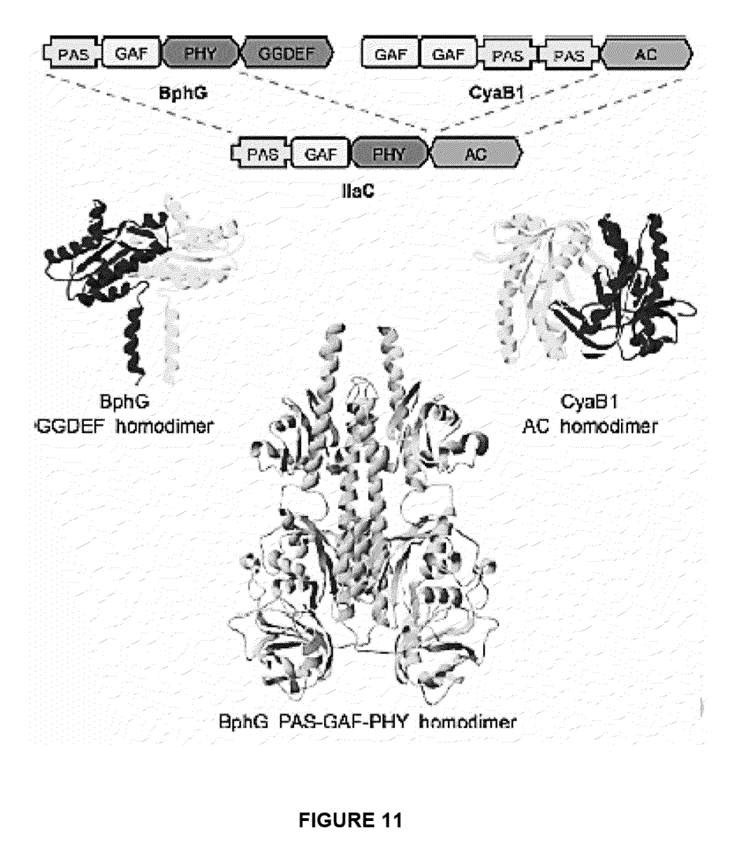

Most or all bacteriophytochromes function as homodimeric enzymes, usually histidine kinases and, more rarely, diguanylate cyclases (DGCs). Enzymatic activities of both histidine kinases and DGCs require precise alignment of two monomers in a homodimer. In case of DGCs, their product, cyclic dimeric GMP (cdi-GMP), is synthesized from two GTP molecules at the interface between two GGDEF domains (Pfam database; [Punta et al., 2012]) responsible for DGC activity. Each GGDEF domain brings a substrate molecule to the catalytic site (Schirmer and Jenal, 2009; Rdmling et al., 2013). In the inhibited state, the photosensory modules were shown to prevent enzymatic domains from forming a properly aligned homodimer, while light-induced conformational changes restore enzymatically productive domain alignment. We believe the conformational changes are mediated by the α-helical linkers that connect the photosensory modules to the output domains (Yang et al., 2009; Yang et al., 2011) (FIG. 11). Further, in all DGCs whose regulation has been studied at the structural level, enzyme activation has been shown or predicted to occur via alignment of the rigid GGDEF domains rather than via intradomain conformational changes (Schirmer and Jenal, 2009; Romling et al., 2013). Because of these considerations, bacteriophytochromes can regulate diverse output activities that depend on proper alignment of the two output domains.

Here we demonstrate that bacteriophytochrome photosensory modules can indeed regulate heterologous output domains.

Earlier, we and others described naturally occurring blue-light activated ACs whose utility in optogenetic applications in cell cultures and small animal models has been demonstrated (Schroder-Lang et al., 2007; Ryu et al., 2010; Stierl et al., 2011; Weissenberger et al., 2011; Efetova et al., 2013). However, undesirable effects of blue light and its poor penetration through nontransparent tissues present major obstacles for the use of blue-light activated ACs, which can be solved by IlaCs. The teachings hereof make it possible for those of ordinary skill in the art to engineer new NIRW light-activated optogenetic tools.

### EMBODIMENTS

Methods are provided herein for producing photoactive fusion proteins based on photoreceptor modules of bacteriophytochromes having a desired activity controllable by near-infrared (NIR) light, said methods comprising the steps:

a. identifying, based on biochemical information, candidate protein output domains that function as homodimers and can be activated by homodimerization;
b. using 3D structures or building 3D models to identify optimal fusion sites and peptide linkers for attaching the heterologous output modules to the bacteriophytochrome photoreceptor module;
c. producing a plurality of DNA molecules (a DNA library), each encoding a monomer of the homodimeric fusion protein that has at least one unique fusion site or linker sequence;
d. screening the DNA molecules for their ability to produce homodimeric photoactive fusion proteins capable of performing the desired activity in a test organism, wherein the screening is done by transforming the test organism designed to respond to the desired activity with the DNA library; allowing the expressed fusion proteins to spontaneously bind biliverdin IXα and form homodimers; applying light of selected wavelengths to the transformed organisms; and comparing the level of said desired activity of the expressed fusion proteins in the test organism in the dark and in the light;
e. optionally subjecting fusion proteins that have NIR light-activated activities identified by screening to random mutagenesis and subsequent screening (as described above) for mutant derivatives with improved qualities, e.g., low activity in the dark and high photoactivation ratio; and
f. purification, and spectral and biochemical characterization of fusion proteins produced by screening to assess their activity levels and photoactivation ratios in vitro.

Candidate output activity to be regulated by NIR light resides within a homodimeric protein. Desired output activity is revealed upon homodimerization, while monomeric output domains should have no or low, background, activity.

Analysis of existing 3D structures and structure modeling of proteins having a desired activity can be performed to identify suitable output modules. The N-terminal boundaries of the functional output domains are defined, and a distance between the N-terminal boundaries is estimated based either on 3D structures or models of 3D structures. This distance is compared to the distance between the C-termini of α-helices extending from the PHY domains of the bacteriophytochome photoreceptor module (PAS-GAF-PHY) homodimer that will be used for fusion. These distances need to be within several angstroms (Å) from each other. Should the distances deviate by more than approximately 10 Å, prior to designing fusion sites, adjustments are made by increasing or decreasing the length of α-helixes extending from the PHY domains of the bacteriophytochome photoreceptor module. Said adjustments will change the distance between the C-termini of α-helixes to better match (within several A) the distance between the N-terminal boundaries of the functional homodimeric output domains. Structures of many proteins having desired activities, protein 3D structure modeling approaches and software are known to the art. Extension of α-helixes may rely on native sequence of the bacteriophytochrome protein or on artificial amino acid sequences known to form α-helixes. Should modification of the lengths of α-helixes extending from the PHY domains be insufficient for bringing the distances between said α-helixes and the N-terminal boundaries of the homodimeric output domains in the proximity of several A, positions of the N-terminal boundaries can be adjusted, i.e., shortened or extended, provided that such adjustments preserve activity of the homodimeric output modules. Prior to constructing fusion proteins, activities of homodimeric output modules are verified in vitro.

Once the fusion site is chosen, a fusion encoding the chimeric protein is made and tested for desired activity and photoactivation ratio. Typically, a plurality of fusions (a DNA library) is made where the N-terminal position of the output domain is fixed, while the α-helical linkers extending from the PHY domain of the bacteriophytochrome photoreceptor module are made to differ from each other by a single amino acid. Once a fusion protein having the desired NIR light-activated or NIR light-inactivated activity is identified, it has been found that shortening or lengthening the α-helices extending from the PHY domain by one or two α-helical turns will form additional proteins that are also light-activated (or light-inactivated). An α-helical turn, approximately 3.6 amino acids, can be approximated by 3 and 4 amino acid extensions and deletions.

The bacteriophytochrome photoreceptor module that provides sensitivity to light in embodiments is a photoreceptor module from the Rhodobacter sphaeroides BphG1 protein comprising PAS-GAF-PHY domains. The photoreceptor module binds its chromophore, a biliverdin IXα, in vivo and in vitro due to intrinsic biliverdin ligase activity.

The output module can be selected from enzymes and other proteins that have a desired biological activity, e.g., enzymatic activity, or ability to bind DNA, RNA or other proteins. In embodiments, the output modules can include protein kinases, proteases (including caspases), nucleotidyl cyclases, nucleases (including recombinases), DNA-binding and RNA-binding protein modules, and others that are activated by homodimerization.

Some photoactive fusion proteins can be activated or their activity can be enhanced by the application of light of an activating wavelength. They can be inactivated, or their activity can be reduced by the absence of light or by the application of light of an inactivating wavelength. Some photoactive proteins can be active or show enhanced activity in the dark or reduced light, and be inactivated or show reduced activity when light of an inactivating wavelength is applied. The “absence of light” can mean the absence of all light (i.e., darkness), or can mean the absence of light in a selected wavelength range that causes a change in the conformation of the bacteriophytochrome photoreceptor module.

In embodiments, in which the fusion protein is in a stable active form (i.e., the output module is in a conformation such that it performs a desired activity when no NIR light is applied), when NIR light of a first wavelength is applied, the conformation of the output module changes and the output module immediately becomes inactive. In such embodiments, the inactive state is relatively unstable. When NIR light of a second wavelength is applied to the fusion protein, it immediately reverts to the stable, active form. If light of the second wavelength is not applied, then the fusion protein gradually reverts to its stable, active form.

In embodiments, in which the fusion protein has a stable inactive form, the opposite is true: the fusion protein is inactive until NIR light of a first wavelength is applied. Then it immediately becomes active. It can be immediately inactivated by application of NIR light of a second wavelength or it can be gradually inactivated by not applying NIR light of the second wavelength.

Thus, in embodiments the desired activity is increased by the application of NIR light of a selected wavelength. In embodiments the desired activity is decreased by the application of NIR light of a selected wavelength. In embodiments the desired activity is gradually decreased or gradually increased by ceasing to apply NIR light of a selected wavelength. In embodiments the desired activity is immediately increased or decreased by the application of NIR light of a selected wavelength. Suitable selected wavelengths are determined by the spectral properties of the bacteriophytochrome photoreceptor module and readily ascertained by those of ordinary skill in the art without undue experimentation.

It is to be understood that the terms “active” and “inactive” in the foregoing explanation are relative and include complete activity of the protein to complete inactivity of the protein (complete “on/off” modes) as well as relative activity or inactivity of the proteins, i.e., the fusion proteins can have high activation ratios, low activation ratios, or activation ratios between high and low. In embodiments the fusion proteins can be controlled by light to have high ratios of activity to inactivity or of inactivity to activity under the control of light of appropriate wavelengths. High ratios are defined herein as ratios of about 2:1 or greater, in embodiments, about 5:1 to about 10:1 or greater. Low ratios are less than about 2:1.

In embodiments, the fusion proteins to be screened can be produced in test organisms already having endogenous chromophore molecules that will bind with the fusion proteins as they are expressed.

In embodiments where no or insufficient chromophore molecules are endogenously available in the test organisms, in addition to producing DNA molecules encoding the designed fusion proteins and expressing them in test organisms, DNA encoding a heme oxygenase can also be expressed in the test organisms, e.g. Rhodobacter sphaeroides heme oxygenase BphO1 (RSP_{—}4190) (Tarutina et al., 2006). The heme oxygenase degrades heme that is present in the test organisms to produce biliverdin IXα chromophore, which then binds to the expressed fusion proteins and make them photoactive. The DNA encoding the fusion proteins can be introduced into the test organisms on the same expression cassette as the DNA encoding heme oxygenase. Suitable expression cassettes comprising DNA for expression under control of appropriate regulatory elements such as promoters are known to the art.

Test organisms for use herein can be any organisms known to the art in which the level of the desired activity can be detected, including cultured organisms selected from the group consisting of E. coi, yeast, plant, or animal cells selected or modified so as to detectably exhibit the level of activity of the expressed fusion proteins under control of NIR light.

When using the fusion proteins produced by the present methods to treat living cells or organisms by controlling processes in these cells or organisms, there can be sufficient endogenous chromophores in the organisms to bind with the expressed fusion proteins, or if not, the organisms can be transformed with a heme oxygenase gene that will be expressed to produce heme oxygenase, which degrades heme that is present in the organisms to produce the chromophore molecules that will bind with the expressed fusion proteins in vivo.

In embodiments, additional fusion proteins controllable by NIR light can be produced by mutagenizing genes encoding “first-generation” NIRLAHPs to create fusion proteins that have lower background activities and higher photoactivation ratios. Mutagenesis was found to improve such protein parameters when applied to DNA encoding the α-helical region linking the PHY domain with the output domain, as well as when applied to the full-length gene encoding a fusion protein.

Thus, fusion proteins that are found to be controllable by NIR light can be the basis for designing additional candidate fusion proteins by mutagenesis and repeating the steps of producing DNA encoding the additional fusion proteins, transforming suitable organisms with this DNA, expressing the DNA, and screening the resultant fusion proteins for additional fusion proteins controllable by NIR light. DNA molecules encoding such additional designed fusion proteins are then made, expressed in test organisms, and screened for their levels of the desired activity.

To further enhance the photoactivation ratios of fusion proteins, the second generation fusion proteins generated by mutagenesis of the first-generation fusion proteins can be mutagenized further to create improved NIRLAHPs. DNA molecules encoding such further designed fusion proteins are then made, expressed in test organisms, and screened for their levels of the desired activity.

The methods hereof comprise selecting or constructing a suitable organism for producing and screening the plurality of DNA (DNA library) encoding NIR light-activated fusion proteins. Any suitable organism known to the art for expression of fusion proteins can be used, so long as the level of the desired activity of the proteins in the organism can be detected. In embodiments, the level of the desired activity can be directly monitored by means known to the art, e.g., by detecting the blue color of 3-galactosidase when it is a marker for a protein produced as the desired activity of the fusion protein. In embodiments, the test organism can be modified as is known to the art to allow detecting of the desired activity of the fusion protein. For example, the test organism can be engineered to allow detection of the desired activity by mutagenesis to prevent it from producing a substance that it would normally produce, so that it can only produce this substance if it expresses an active fusion protein.

The photoactive fusion protein can have any activity known to the art. Typically the activity involves control of a process in vivo such as a metabolic process, signal transduction, cell apoptosis, cell proliferation, cell adhesion, or cell differentiation. In embodiments, the photoactive fusion protein is selected from the group consisting of a light-activated nucleotidyl cyclase, such as adenylyl cyclases (also known as adenylate cyclases) or guanylyl cyclase (also known as guanylate cyclases), and a light-activated uncleavable procaspase-3.

NIR photoactive fusion proteins are also provided herein. Such proteins can be produced by the methods described above, or by methods analogous thereto that can be designed and carried out by those of ordinary skill in the art without undue experimentation.

Further provided herein are recombinant DNA molecules encoding the homodimeric fusion proteins described herein. Expression cassettes comprising such DNA molecules under control of appropriate regulatory elements are also provided.

Also provided herein are methods for controlling an in vivo process in a host, which is a living cell or organism. The method comprises:
a. introducing into the cell or organism or selected portion of the organism a DNA sequence encoding a homodimeric fusion protein comprising a bacteriophytochrome photoreceptor module and a heterologous output module capable of modulating the desired process;
b. introducing into the cell or organism a DNA sequence encoding a heme oxygenase capable of producing biliverdin IXα, if the endogenous level of biliverdin IXα in the cell or organism is insufficient for photoactivation;
c. providing a source of heme (the substrate for heme oxygenase), if the host cell or organism does not contain sufficient endogenous levels of heme; or providing biliverdin IXα;
d. allowing the fusion protein and heme oxygenase, where applicable, to be expressed in the host; and
e. applying NIR light of a selected wavelength to the host or preventing NIR light of a selected wavelength from reaching the host; thereby modulating the process under control of NIR light.

Methods of introducing DNA into selected portions of organisms are well-known to the art. Methods for selectively expressing DNA in chosen portions of an organism are also well-known to the art, including use of tissue-specific promoters. These techniques can be combined with the application of light to selective portions of an organism to control expression of the homodimeric proteins hereof in desired tissues and organs.

The in vivo process can be selected from the group consisting of metabolic processes, signal transduction, cell apoptosis, cell proliferation, cell adhesion, and cell differentiation.

The photoreceptor module can be as described above, e.g., that of Rhodobacter sphaeroides BphG1 protein.

### DETAILED DISCUSSION

The engineering principles disclosed herein are applied to select and optimize NIR light-activated homodimeric proteins (NIRLAHPs). These proteins can be used to turn on (or turn off) desired activities in transgenic animals, plants or microbes.

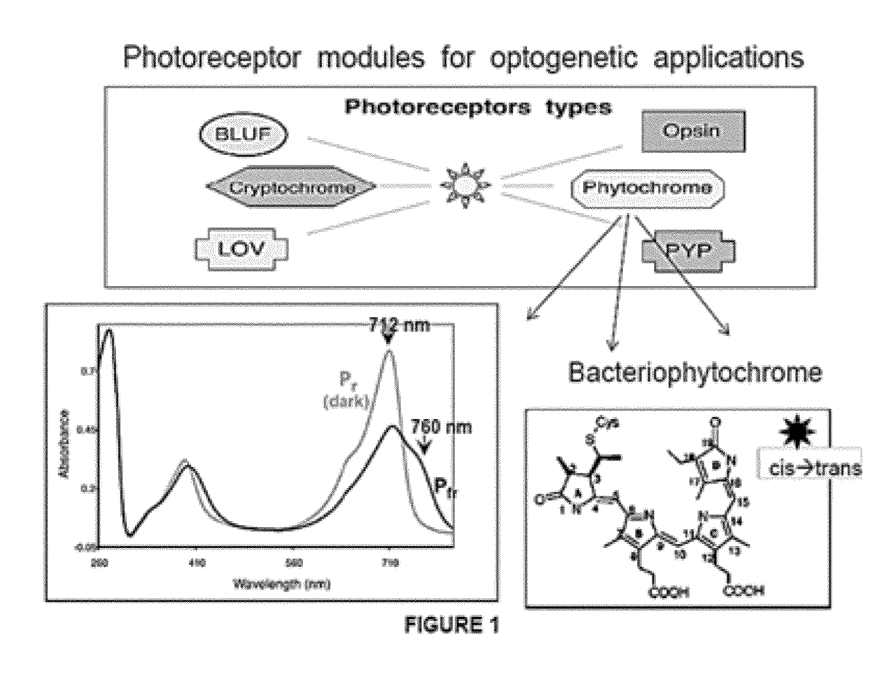

Bacteriophytochromes can significantly expand the range of optogenetic applications: (i) They absorb light of the far-red/NIR spectrum (Rockwell et al., 2006, FIG. 1B), which penetrates animal tissues much deeper than visible light sensed by currently used photoreceptors (Cuberddu et al., 1999; Wan et al., 1981; Byrnes et al., 2005). (ii) NIR light is harmless; for example, it is currently used in human optical imaging and deep-tissue phototherapies (Fang et al., 2009; Desmet et al., 2006). (iii) The biliverdin chromophore of bacteriophytochromes is the first product of heme breakdown and thus is naturally produced by most animal cells (Rockwell et al., 2006). If insufficient, biliverdin (which is nontoxic in small doses) can be directly injected (Shu et al., 2009) or supplied by a bacterial heme oxygenase. (iv) Phytochromes can be instantly turned “off” (i.e., photoinactivated) by longer wavelength light (Rockwell et al., 2006), which provides for excellent temporal control. (v) Lastly, recently (far-red light absorbing) phytochrome-based fluorescent proteins have been expressed in mice and used for whole-body imaging, which proves that phytochromes expressed in deep tissues can be activated by external light sources in small mammals (Shu et al., 2009). In sum, NIR light-activated proteins can significantly broaden the range of optogenetic applications and allow researchers to use these approaches in the mammalian models of development and diseases as well as in other organisms.

Bacteriophytochromes function as homodimers. The light-induced conformational changes in the photosensory module of one monomer are presumed to rotate its output domain and bring it into proximity with the output domain of the second monomer, thus generating an active conformation of the homodimer. Natural bacteriophytochromes have different homodimeric outputs, e.g., His-kinases and diguanylyl cyclases.

Any bacteriophytochrome can be used in the methods and homodimeric proteins provided herein provided it is responsive to light. A few proteins classified as bacteriophytochromes may not respond to light. Responsiveness to light can be tested by those of ordinary skill in the art without undue experimentation.

In addition, those of ordinary skill in the art can determine the most effective wavelengths for controlling the activity of the homodimeric proteins provided herein without undue experimentation by means known to the art and described herein, such as spectroscopically observing differences between the spectra of the homodimeric proteins in light and dark (Rockwell et al., 2006).

This disclosure illustrates engineering of photoactivated versions of nucleotidyl cyclases and executioner caspase. Engineering principles for constructing NIR light-activated homodimeric proteins are provided. Since a large number of signaling proteins function as homodimers, NIR light-induced protein homodimerization can be used to control a variety of cellular functions including metabolic processes, signal transduction, cell apoptosis, differentiation, proliferation, transformation and adhesion.

This disclosure also illustrates the use of the homodimeric proteins hereof for controlling neuronal activity in the roundworm Caenorhabditis elegans. The homodimeric proteins hereof can be used to control other in vivo processes as described above, for example through the use of light-activated adenylate cyclases to control production of cAMP, which in turn controls are wide range of metabolic processes. See, e.g., Chin et al., 2002.

In addition, the proteins can be used to control metabolic processes in a wide range of living organisms. Bacteriophytochrome photosensors have also been used to engineer monomeric fluorescent proteins expressible in mammals (Shu et al., 2009). The expression of the engineered homodimeric proteins provided herein can be applied to any desired cell or organism by one skilled in the art without undue experimentation using the teachings hereof as well as art-known techniques of molecular biology. Specific techniques for transformation applicable to specific cells and host organisms, are well-known to the art. In addition, methods of introducing cells capable of expressing the homodimeric proteins hereof into host organisms are well known to the art. Those of ordinary skill in the art can determine effective levels of expression to accomplish desired metabolic results without undue experimentation using art-known knowledge and the teachings herein.

The methods and engineered homodimeric proteins provided herein can be used in research investigating the pathways, neural and chemical involved in various metabolic functions in vivo, and for treatment of various disease conditions including cancer, neurological and cardiac conditions and other diabetes and other hormonal dysfunctions. It can also be used in industrial biological processes to control the output of desired products.

This disclosure illustrates engineering of NIRLAHPs using the BphG1 protein from R. sphaeroides. However, numerous bacteriophytochromes are present in the genomes of microbes, primarily in bacteria. Because they likely undergo similar light-induced conformational changes to those that occur in BphG, these bacteriophytochromes can also be used as sources of photoreceptor modules for protein engineering.

Construction of photoactivated fusions starts with identification of output activities known to be activated by homodimerization. Subsequently, analysis of 3D structures (or structural models) of the photosensory module and homodimeric output module is undertaken. A fusion point for creating photoactivated chimeric proteins is based on using approximately the same distance (in three-dimensional space) between the C-termini of the α-helices extending from the PHY domains of the homodimeric photosensory modules as the distance between the N-termini of the homodimeric output modules. These distances are derived from 3D structures (X-ray and NMR) or structural models built based on 3D structures. The α-helices extending from the PHY domains can be shortened or extended to accommodate the N-termini of the output module. The fusions can occur at different boundaries of the output module; therefore, several fusion sites are tested to identify fusion proteins with optimal parameters, i.e., high photoactivation ratio (the ratio of protein activity in the light to that in the dark, also known as dynamic range) and low activity in the inactive state (which is the dark state for photoactivated proteins, or lit state in the photoinactivated proteins). Our analysis of an engineered NIRLAHP-adenylyl cyclases (where the output module is the adenylyl cyclase domain of the CyaB1 protein from Nostoc sp.) suggests that the light-induced conformational changes in the photosensory domain of a bacteriophytochrome monomer result in a movement, that may involve rotation, of its output domain that brings it in proximity with the output domain of the second monomer, thus generating an active homodimer.

The relative positions of the output domain monomers depend on the phase of the α-helices that link the PHY domains of the photosensory module to the output domains. The output domains that are linked on the same side of the α-helices display similar light responsiveness. For example, several light-activated fusion proteins have been obtained that differ from each other by multiples of 3 or 4 residues, which corresponds to one, two or more α-helical turns, where one α-helical turn is approximately 3.6 amino acid residues. The torque generated by the presumed rotation of the photosensory module following photon absorption is believed to change mutual arrangement (possibly via rotation) of the output domains. For transfer of the torque to the output domains, unstructured elements (e.g., loops) preceding the more rigidly structured elements of the output domains should be minimized.

Once a first-generation NIRLAHP is obtained, its photoactivation ratio can be improved via mutagenesis (e.g., via error-prone PCR mutagenesis using the whole fusion protein as a template at the rate of several mutations per gene, or via integration of degenerate synthetic DNA sequences).

NIRLAHPs possessing lower dark activities and higher photoactivation ratios, compared to the first-generation NIRLAHPs, can be identified following the same mutagenesis and screening procedures.

Selection and/or screening for the first-generation NIRLAHP of its class as well as identification of mutants with maximal photoactivation ratios can be achieved by using specifically designed microbial or animal cells. For example, screening for a light-activated adenylyl cyclase is done in the E. coli mutant impaired in the cya gene that encodes a native adenylyl cyclase.

Cyclic nucleotides are universal second messengers that control various important biological processes. However, precise roles of cAMP and cGMP in many physiological processes and diseases remain unknown. A number of drugs for chronic obstructive pulmonary disease, bone marrow transplant rejection, and cancer increase cellular cAMP, which in turn decreases inflammation (reviewed in Serezani et al., 2008). Some of the primary signals inducing cAMP synthesis in cells include epinephrine, norepinephrine, histamine, serotonin, and certain prostaglandins (Landry et al., 2006). The photoactivated adenylyl cyclase allows understanding of signaling pathways with higher precision than that provided by the use of hormones. Blue-light-activated adenylyl cyclase from Euglena gracilis (Iseki et al., 2002) and Beggiatoa sp. (Ryu et al., 2010; Stierl et al., 2011) can be applied to study various biological processes in cell cultures and animals transparent to light, e.g. zebrafish. The NIR light version of adenylyl cyclases allows researchers to study cAMP-signaling in both transparent model organisms and, importantly, organisms that are non-transparent to visible light, e.g. red-blooded animals.

The near-infrared light version of guanylyl cyclase can be made by site-directed mutagenesis of as few as 2-3 amino acid residues in the adenylyl cyclase (ACyc) domain as known in the art (Ryu et al., 2010)

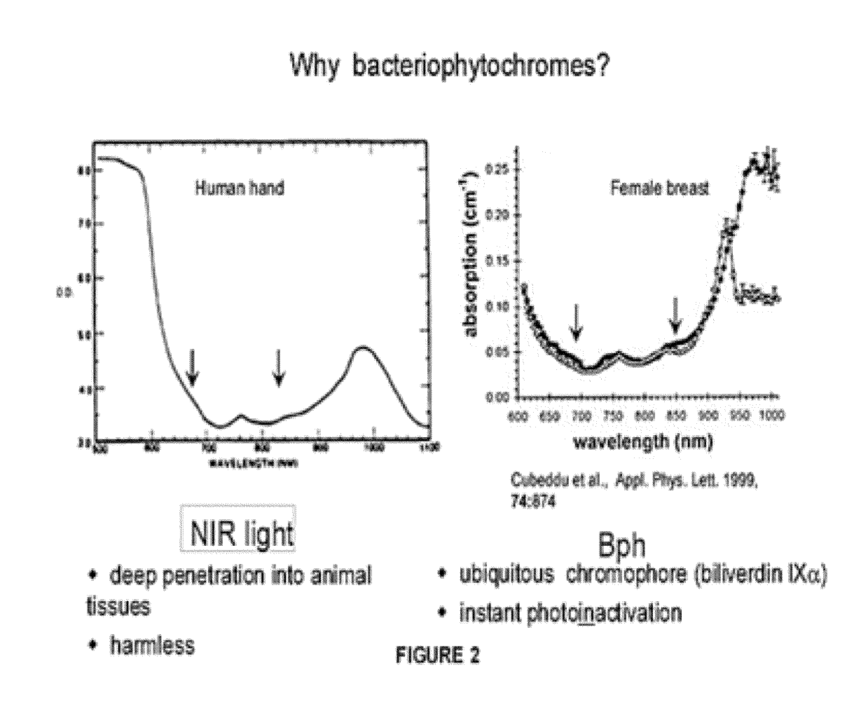

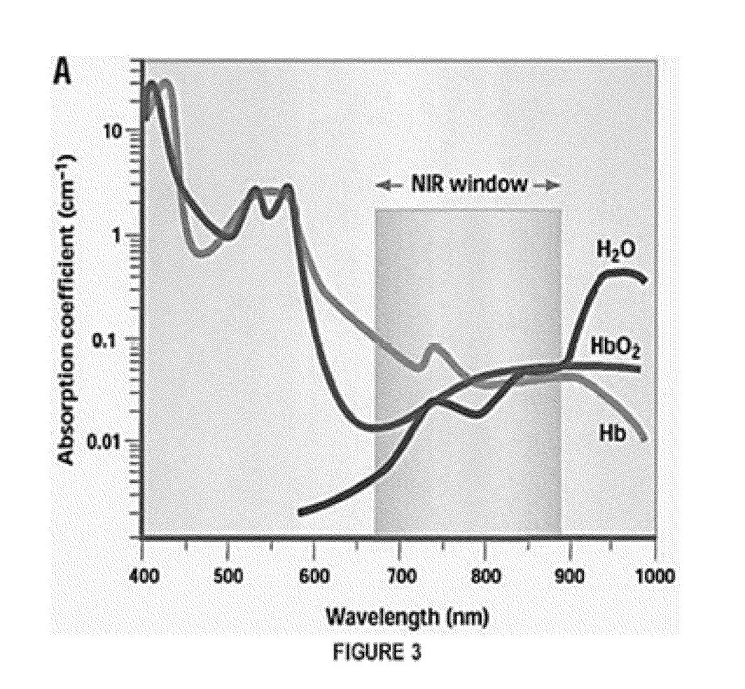

Photoactivated caspases, are another biological tool disclosed herein. They allow researchers to conduct targeted cell/tissue killing in vivo using NIR light, and are applicable in many areas of biology and medicine, particularly in tumor biology, immunology and developmental biology. Currently-available approaches that target cells for killing, e.g., laser ablation, and chromophore-assisted light-inactivation with chemical or genetically encoded photosensitizers (Jacobson et al., 2002; Bulina et al., 2006), are harsher (i.e., damage nearby cells/tissues), less precise and/or poorly applicable to mammalian models. A photoactivated caspase, whose gene can be delivered in tumors (e.g., by recombinant viruses, bacteria or nanoparticles), can be used as a readily controllable cancer gene therapy. It can be used in isolation or in combination with already-existing cancer treatments (e.g., cytotoxic drugs). A blue-light activated executioner caspase-7 has recently been engineered and shown to efficiently kill cells in cell culture in response to blue light (Mills et al., 2012). However, the utility of blue-light activated caspase, as well as other blue-light activated proteins is limited in red-blooded animals because of low light penetration through animal tissues (FIGS. 2, 3). Therefore, a NIR-activated caspase represents a transformative improvement that enables its use in animal models of disease and development.

Engineering and Optimizing Near-Infrared Light-Activated Nucleotidyl Cyclases.

Cyclic nucleotides are universal second messengers that control a variety of processes including cell growth and differentiation, blood glucose levels, cardiac contractile function, learning, memory, and other processes known to the art. The ability to activate cAMP and/or cGMP synthesis in desired cells at specific development/disease times is used to provide new and important mechanistic insights into cyclic nucleotide signaling.

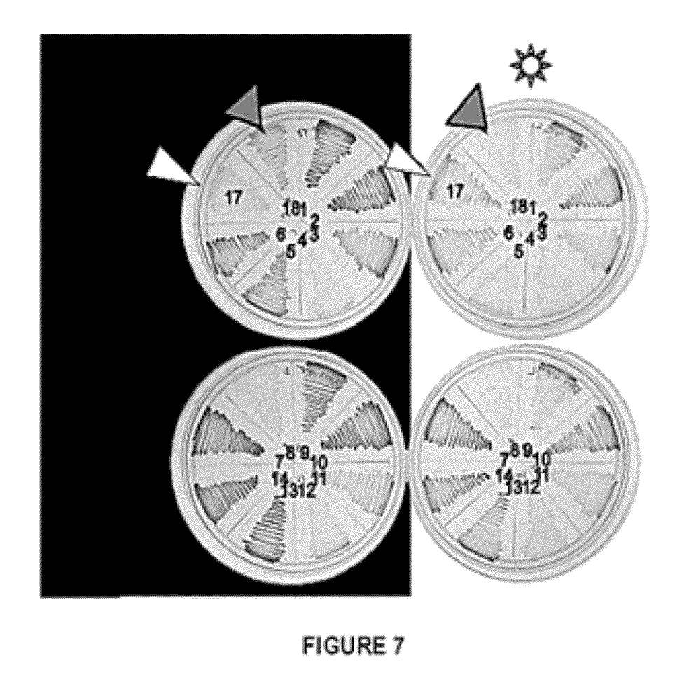

As shown herein, bacteriophytochrome photosensory modules were engineered to activate heterologous outputs. In an embodiment hereof, to construct NIR light-activated nucleotidyl cyclases, the diguanylyl cyclase GGDEF domain from the photoactivated diguanylyl cyclase, designated BphG, from Rhodobacter sphaeroides was replaced with a distantly related adenylyl cyclase (ACyc) domain from Nostoc sp. protein CyaB1 resulting in the production of photoactivated adenylyl cyclase, designated RlaC (FIG. 7-9). The first-generation adenylyl cyclases were mutagenized to identify variant enzymes with the highest photoactivation ratio and lowest activities in the dark. Optimized adenylyl cyclase is used as a template to engineer a NIR light-activated guanylyl cyclase as described by Ryu et al. (2010).

Engineering and Optimizing Near-Infrared Light-Activated Executioner Caspases.

Executioner (effector) caspases are terminal cysteine proteases initiating apoptosis (programmed cell death). An engineered photoactivated executioner caspase is useful to induce apoptosis in desired cells or tissues of recombinant animals expressing it in specific tissues. A gene for a photoactivated caspase can also be delivered to tumors and used in noninvasive cancer gene therapy. In an embodiment hereof, a derivative of the executioner caspase, procaspase-3, which is activated by homodimerization, is engineered using principles developed from engineering and optimizing near-infrared light-activated nucleotidyl cyclases to construct a near-infrared light-activated caspase. All engineered enzymes are biochemically characterized in vitro. Prioritized constructs are moved into Drosophila melanogaster, mice and other organisms.

The following description of various specific embodiments is exemplary in nature and is in no way intended to limit the scope of the claims hereof. In embodiments, art-known equivalents of exemplified components, materials and method steps can be substituted for those specifically described herein and these embodiments are considered to fall within the scope of the claims. Embodiments including less than all the components, materials and method steps of embodiments specifically described herein are also considered to be encompassed within this disclosure.

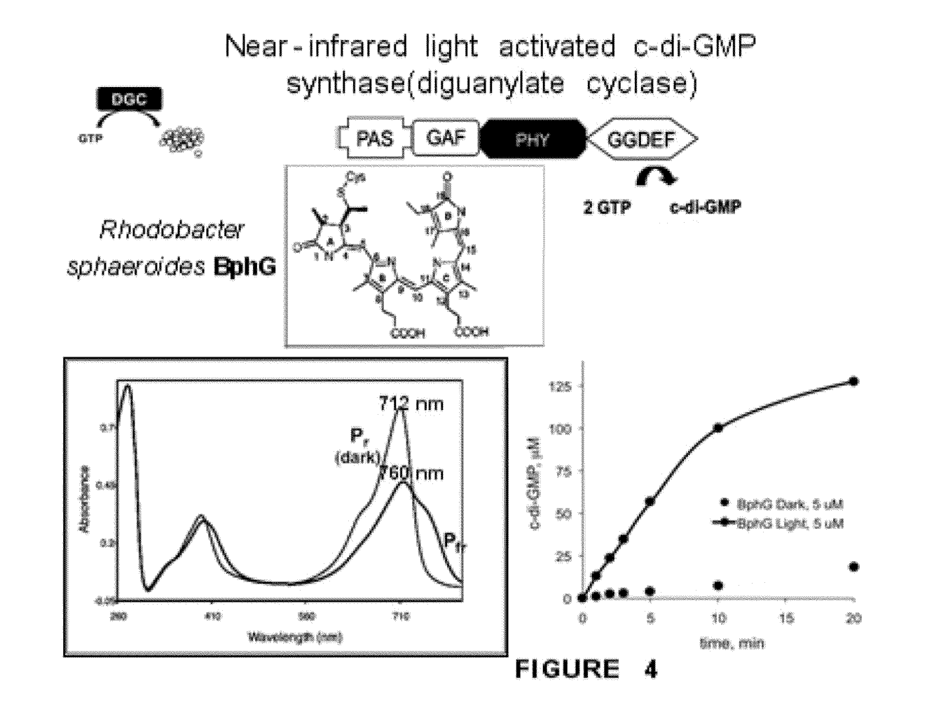

FIG. 1 (top panel) depicts six major types of photoreceptors (molecules that organisms use to detect light): opsins, which are human retinal photosensors and rhodopsins of various microbes; cryptochromes, which are blue light-sensitive flavoproteins found in plants, animals and microbes; photoactive yellow protein (PYP) photosensors, which are found in certain bacteria; photoreceptors of blue-light using flavin adenine dinucleotide (BLUF) and Light, Oxygen, or Voltage sensing (LOV) types, which are plant and bacterial photoreceptors; and phytochromes, which are used by plants and microbes and are sensitive to light in the red-to-NIR region. Work done to illustrate the presently-claimed methods was done using a bacteriophytochrome, a subclass of phytochromes that covalently bind biliverdin IXα as a chromophore (a molecule bound to the photoreceptor protein that detect slight and cause a conformational change in the protein when hit with a photon of light). The bacteriophytochrome (Bph) used herein was Rhodobacter sphaeroides BphG, which converts two guanosine triphosphate (GTP) molecules into cyclic dimeric guanosine monophosphate, c-di-GMP. As shown in the lower left panel of FIG. 1, the dark form of BphG, has a protein conformation designated Pr, which is present in the dark or absence of 650-715 nm light. When light having a wavelength between about 650 and about 715 nanometers strikes the chromophore, it causes a rearrangement of the molecule to an isomeric form designated as far-red, Pfr, conformation, in which the double bond (between C15 and C16) shifts from the cis to trans conformation. The protein absorbs light maximally at 712 nm resulting in the red-shifted, Pfr, form, whose diguanylyl cyclase activity is approximately 10-fold higher compared to the activity of the Pr form, as shown in FIG. 4. The chromophore of the bacteriophytochrome has the chemical structure depicted in the lower right panel of FIG. 1.

Once formed upon irradiation, the Pfr form of BphG is fairly stable. In the dark it spontaneously converts to the Pr form in approximately 45 min (Tarutina et al., 2006). If light of about 760 nm is applied, the Pfr form is converted to the Pr (dark) instantly. This reversible photoconversion feature is a unique to phytochromes.

The present methods are especially useful for application in humans and other mammals because mammalian flesh is relatively transparent to far-red/NIR light. As shown in FIG. 2, left panel the absorption of light by a human hand decreases as the wavelength of the light changes from blue to far-red and NIR light, being at its lowest between about 680 to about 890 nm, in the so-called “NIR window” (FIG. 3). FIG. 2, right panel shows absorption of light in female breast tissue, again being minimal in the NIR window. The light in the NIR window can penetrate deeply into the body (many centimeters). The light in the NIR window is harmless because there are no chromophores in animals that absorb in the NIR window.

The main advantages of bacteriophytochromes in use in optogenetics are that their chromophore, biliverdin IXα, is made in mammals, where, as indicated in FIG. 2, it is produced as a natural breakdown product of heme. For use of the chimeric proteins hereof in mammals, there is no need for a step of administering the chromophore separately. Bacteriophytochromes can be instantly photoinactivated, e.g., by applying light of 750-780 nm for BphG. This provides for superior, compared to other photoreceptor types, temporal regulation of output activities of chimeric bacteriophytochromes.

In embodiments when using the fusion proteins hereof for treating an organism, the organism will produce sufficient chromophore molecules for effective use of the NIR-light-controlled fusion protein. However, if biliverdin Ixα is insufficient in a particular tissue or animal model, it can be administered externally (it is nontoxic to animals at low doses), or it can be synthesized by heme oxygenase that can be delivered as a gene on the same gene delivery platform as the chimeric bacteriophytochrome.

FIG. 3 shows the “NIR window” from about 670 nm to about 890 nm, where cumulative absorption by three major light-absorbing components of flesh in red-blooded animals, deoxyhemoglobin (Hb), oxyhemoglobin (HbO₂) and water (H₂O), is lowest (Weissleder, 2001). The “NIR window” identifies the range of wavelengths that can be used for deepest penetrating through mammalian tissues. Bacteriophytochrome absorption peaks fall into the “NIR window”.

FIG. 4 illustrates protein domain architecture (top panel) as well as spectral and enzymatic properties (bottom panel) of the BphG protein used for protein engineering. Top panel: R. sphaeroides BphG (PAS-GAF-PHY-GGDEF) is a derivative of BphG1 (PAS-GAF-PHY-GGDEF-EAL) lacking the EAL domain (Tarutina et al., 2006). BphG converts two guanosine triphosphate (GTP) molecules into cyclic dimeric GMP (c-di-GMP) by means of a diguanylate cyclase activity of the GGDEF domain (Ryjenkov et al., 2005). Bottom panel, left: Spectral properties of BphG (same as in FIG. 2). Bottom panel, right: Synthesis of c-di-GMP by R. sphaeroides in vitro, in the dark (grey line) and in the light (black line).

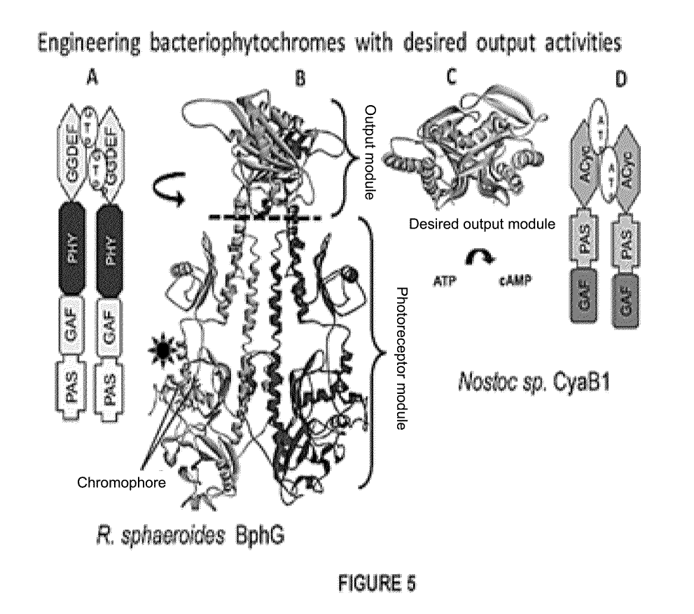

FIG. 5A is a schematic depiction of the R. sphaeroides BphG protein comprising the photoreceptor (PAS-GAF-PHY) and output (GGDEF) modules. The BphG protein is depicted as a parallel homodimer. FIG. 5B is a 3D model of the BphG protein based on the 3D structure 3c2w for the phototreceptor module and 3icl for the output domain (3D structures from Protein Data Bank, PDB, rcsb.org/pdb). The dashed line represents approximate position in the α-helices extending from the photoreceptor domain for fusion with a heterologous homodimeric output module. An arrow indicates rotation of an output domain as a potential outcome of light-induced conformational changes in BphG. FIG. 5C depicts a 3D model of the homodimeric adenylyl cyclase domains of protein CyaB1 from Nostoc sp. (modeled based upon the PDB structure 1wc5). The output module of R. sphaeroides BphG, the diguanylyl cyclase GGDEF domain was replaced with a distantly related adenylyl cyclase (ACyc) domain from Nostoc sp. CyaB1 resulting in the photoactivated adenylyl cyclase. FIG. 5D is a schematic representation of the protein domain architecture (GAF-PAS-ACyc) of Nostoc sp. CyaB1 depicted as a homodimer.

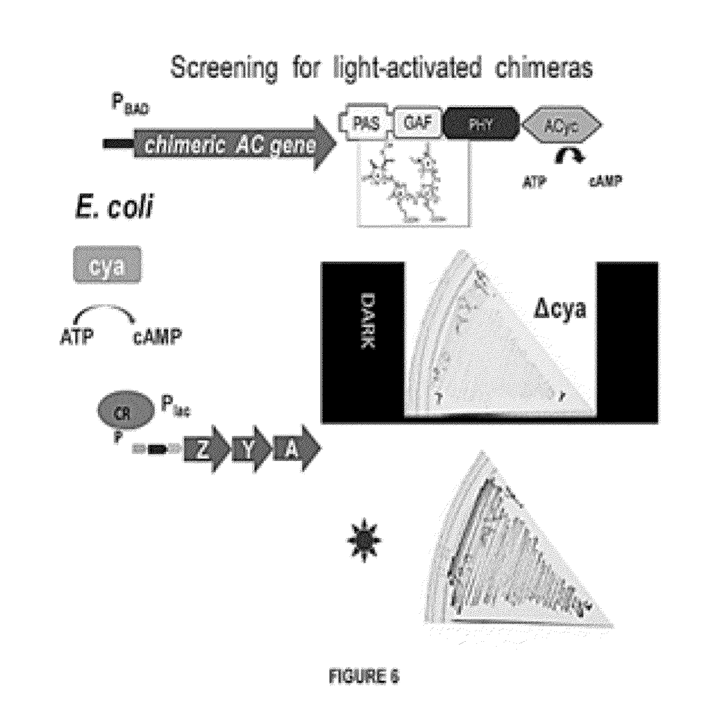

FIG. 6 illustrates the use of E. coli as a test organism to screen a library of fusion protein constructs for adenylyl cyclase activity. A library of fusion proteins encoded by the “chimeric AC (adenylyl cyclase) genes” is expressed (from a P_{BAD} promoter) in the E. coli strain having a cya gene deletion, which does not naturally produce cAMP. If the fusion protein generates cAMP, it enables expression of the (CRP-cAMP-dependent) lacZYA operon, which results in blue colonies on XGal indicator plates. In the absence of cAMP, the colonies are colorless. By comparing the color of colonies grown in the light (650-nm LED panel) and in the dark, one can identify light-activated and light-inactivated versions of adenylyl cyclases. The colonies appearing blue in the light and colorless (or less blue colored) in the dark contain candidate light-activated adenylyl cyclases.

FIG. 7 illustrates XGal indicator plates containing representative E. coli clones, each having a gene encoding a different (numbered) fusion adenylyl cyclase protein construct, grown either in the dark (left panel) or in the light (right panel). Blue color indicates how each clone responds to light. The white arrowhead points to the clone containing a light-activated adenylyl cyclase (SEQ ID NO:6); the black arrowhead points to one of the light-inactivated adenylyl cyclases SEQ ID NO:1).

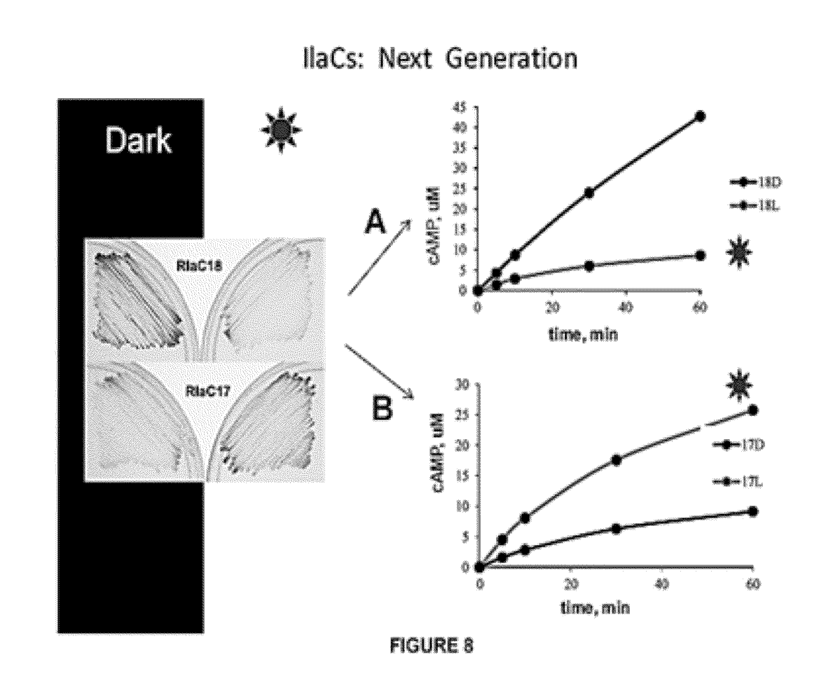

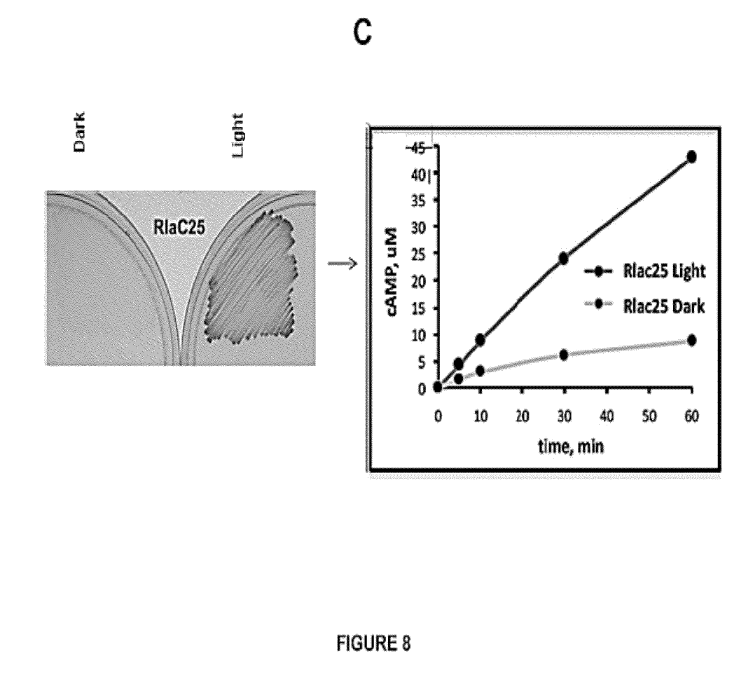

FIG. 8 shows images of E. coli clones containing selected light-activated [(#17, #25)] SEQ ID NO:6, SEQ ID NO:10) and light-inactivated SEQ ID NO:1 fusion adenylyl cyclase proteins grown in the dark and light, along with adenylyl cyclase activities measured using purified proteins under light and dark conditions.

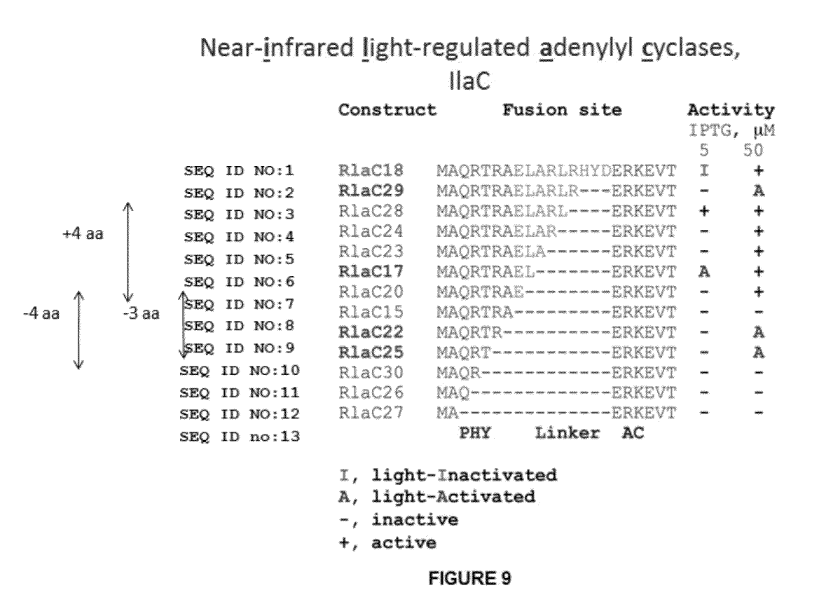

FIG. 9 provides protein sequences near fusion points of selected engineered adenylyl cyclase fusions between the photoreceptor module of BphG and adenylyl cyclase (ACyc domain) of CyaB1. Photoresponses in E. coli of the fusion proteins were expressed at two levels of expression: low (5 μM isopropyl-beta-D-thiogalactopyranoside, (IPTG)) and high (50 μM IPTG). β-galactosidase expression (judged by the intensity of blue color) is dependent on intracellular cAMP levels. A, light-activated (higher β-galactosidase expression in the light versus dark); l, light-inactivated (higher β-galactosidase expression in the dark versus light); +. light-independent activity; −. no activity (in the dark or light). It is emphasized that protein fusions containing approximately one helical turn (+/−3-4 amino acids, aa) longer or shorter linkers had the same type of light-responsiveness, e.g. light-activated SEQ ID NO:6; SEQ ID NO:2 ([#17] SEQ ID NO:6+4 aa); SEQ ID NO:10 (SEQ ID NO:6−4 aa); SEQ ID NO:9 (SEQ ID NO:6−3 aa).

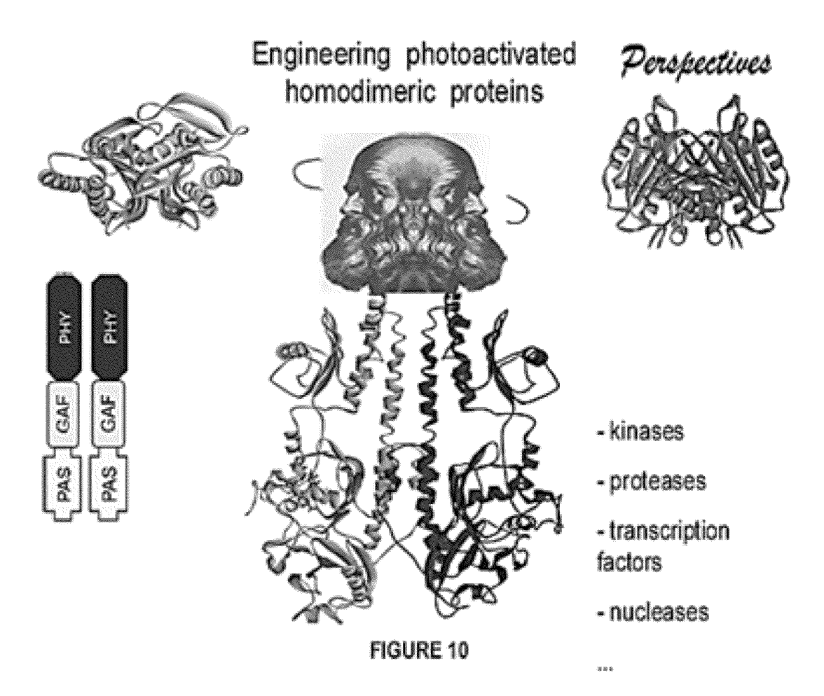

FIG. 10 illustrates an overview of principles elucidated herein. The photosensory module of a bacteriophytochrome is shown schematically in the lower left with a modeled heterologous active module shown above. In the center of the figure, a modeled photosensory module is shown with the active module symbolized by a “homodimeric head.” On the right, a generic modeled active module is illustrated above a list of examples of modules that can be used to engineer NIRLAHPs, e.g., kinases, proteases, transcription factors, nucleases, etc.

To make a chimeric (fusion) protein hereof, one skilled in the art applying the principles taught herein can (1) pick a protein having an activity desired to be controlled by NIR light, that is active in a homodimeric form as two fused monomers, to supply the output module of the fusion protein that is capable of performing the desired activity; (2) provide a photosensory module comprising a bacteriophytochrome, such as that of the BphG protein of Rhodobacter sphaeroides; (3) determine possible fusion sites of the output module and receptor module by matching distances between fusion sites on the output module and the receptor module by lengthening and/or shortening the α-helix linkers of the output and/or photoreceptor module until their fusion sites correspond in space; (4) screen the constructs for light and dark activity; (5) upon identifying active constructs, find additional active constructs by making constructs with α-helical linkers 3-4 amino acids longer or shorter than those of the identified active constructs and screening them for activity; (6) further optimize the performance of the constructs by mutagenesis of either or both of the photosensory and active modules to find fusions that perform better, i.e., low activity in the dark and high activity in the light, or vice versa.

### EXAMPLES

The near-infrared light-activated diguanylyl cyclase from R. sphaeroides, designated BphG, converts two GTP molecules into cyclic dimeric GMP (c-di-GMP) (Tarutina et al., 2006). The dark, Pr, form of BphG absorbs maximally at 712 nm resulting in the red-shifted, Pfr, form (FIG. 4, lower left panel), whose diguanylyl cyclase activity is approximately 10-fold higher compared to the activity of the Pr form (FIG. 4, right panel). To our knowledge, the photoactivation ratio in BphG is the highest among bacteriophytochromes for which such a ratio was measured. This makes BphG particularly attractive for protein engineering. The Pfr form of BphG can be brought back to the ground (dark, Pr) state by irradiation at 750-780 nm (maximum 760 nm, FIG. 4, left panel), which instantly turns the diguanylyl cyclase activity off (Tarutina et al., 2006).

Conformational changes following photon absorption result in the rotation or other movement type in the photosensory module that is transmitted as torque through the α-helixes extending from the PHY domain of the photoreceptor to the output domain of the photoactivated monomer (FIG. 5B). This motion brings two output domains into an active homodimeric state (Yang et al., 2008; Yang et al., 2009). The photosensory module of bacteriophytochromes is capable of activating diverse homodimeric outputs. A two-tiered test of this model was performed. First, we replaced the diguanylyl cyclase GGDEF domain of BphG with a distantly related (˜15% sequence identity) bacterial adenylyl cyclase (ACyc) domain such that the structural relatedness (Pei and Grishin, 2001; Sinha and Sprang, 2006) would increase the chance of a successful domain swap as shown in FIG. 5. Next, we focused on optimizing performance of the photoactivated adenylyl cyclase and constructing a near-infrared light-activated guanylyl cyclase using engineering principles developed in this work to construct a photoactivated homodimeric caspase-3 whose structure is completely unrelated to GGDEF.

We engineered several light-activated fusion proteins that differed from each other by approximately one or two α-helical turns, showing that positioning of the output domains in the same phase of the helix is important for light-dependent activity. Extensive mutagenesis of one of these fusions resulted in an adenylyl cyclase with a six-fold photodynamic range. Additional mutagenesis produced an enzyme with a more stable photoactivated state. When expressed in cholinergic neurons in Caenorhabditis elegans, the engineered adenylyl cyclase controlled worm behavior in a light-dependent manner.

### Example 1

### Engineering and Optimizing Near-Infrared Light-Activated Adenylyl and Guanylyl Cyclases

Engineering a First NIR Light-Activated Adenylyl Cyclase:

For selecting photoactivated adenylyl cyclases, we constructed a cya deletion mutant, E. coli BL21 cya. This strain is devoid of its native adenylyl cyclase (cya mutation) and, therefore, produces white colonies on XGal indicator plates. Plasmid pBphO expressing the R. sphaeroides heme oxygenase, bphO1, which makes biliverdin IXα is introduced in this strain.

We constructed a structural model of the BphG homodimer based upon the most closely related protein structures available in the Protein Data Bank (PDB), i.e., 3c2w for the (PAS-GAF-PHY) photosensory module and 3icl for the GGDEF domain (FIG. 5B) (Kuzin et al., 2009). As a source of the ACyc domain we chose an extensively biochemically characterized cyanobacterial adenylyl cyclase CyaB1. An important consideration for choosing CyaB1 was that its ACyc domains are known to spontaneously dimerize and form homodimers with significant cyclase activity (even in the absence of the regulatory domains) (Bruder et al., 2005). Therefore, once the two ACyc domains are brought in proximity, they were expected to form an active enzyme. The ACyc domain dimer of CyaB1 was modeled based upon the PDB structure 1wc5. See FIG. 5C.

Based on the analysis of distances between the C-termini of the α-helices in the PAS-GAF-PHY homodimer and the N-termini of the ACyc homodimer, an approximate fusion point is chosen (FIG. 9, MAQRTRAERKEVT [SEQ ID NO:8]A library of PAS-GAF-PHY fusions to the ACyc domain of Nostoc sp. CyaB1 was constructed using one fixed site in the ACyc domain (FIG. 9) and variable (by a single amino acid) lengths of the α-helixes extending from the PHY domain of the photosensory module of BphG. These fusions were expressed in E. coli BL21 cya from an IPTG-inducible promoter.

The fusions were plated in the dark with no IPTG, and subsequently screened on a medium containing low and high levels of IPTG, in the absence (foil-wrapped plates) or presence of far-red (650 nm) light provided by LED panels. A set of the fusions with variable length linkers is shown in FIG. 9, where proteins are designated RlaC (red light-activated adenylyl cyclase). We identified four classes of fusion proteins; (a) constitutively active (e.g., RlaC28), (b) constitutively inactive (or nonfunctional, e.g., RlaC15), (c) light-inactivated (e.g., RlaC18), and the desired class of (d) light-activated fusions (e.g., RlaC29, 17, 22, and 25). Analysis of these fusions revealed several important findings. First, we learned that a starting point for creating photoactivated fusions should be based on approximately the same distance (in three-dimensional space) between the helices extending from the PHY domains and between N-termini of the output domains. These distances are derived from structural models (or crystal structures). Second, photoactivated fusion proteins can be obtained at different boundaries of the output domains (however, not all). Therefore, multiple fusion sites should be tested to identify optimal fusions. Importantly, our data are consistent with the signaling helix rotation mechanism of bacteriophytochrome photoactivation. In accord with this mechanism, fusions differing by a complete α-helical turn (i.e., approximately 3.6 aa) position output domains in the same phase of the helix, and thus α-helices differing in length by 3 or 4 amino acids (aa) should have enzymatic activities that respond to light in a similar manner. This is exactly what we observed. For example, all photoactive forms shown in FIG. 9 differ from each other by 3 or 4 aa, i.e., close to a helical turn: RlaC29=RlaC17+4 aa; RlaC25=RlaC17−4 aa, and RlaC22=RlaC17−3 aa (FIG. 9).

First-generation near-infrared light-activated adenylyl cyclases shown in FIG. 9 show useful photoactivation ratios (˜5.5-fold for RlaC25, according to the in vitro activity measurements (FIGS. 7 and 8C). To improve the photoactivation ratios and decrease adenylyl cyclase activity in the dark, error-prone PCR-based mutagenesis using the full-length rlaC25 gene as a template (at the mutation frequency of 3-4 mutations per gene) can be undertaken.

Substrate specificity in class III nucleotidyl cyclases depends on just a few residues (Winger et al., 2008). We have verified this hypothesis by converting the blue-light-activated adenylyl cyclase, BlaC, into a guanylyl cyclase, BlgC (Ryu et al., 2010) using as few as three mutations.

The RlaC and RlgC derivatives are purified and characterized in vitro using methods described by us earlier (Tarutina et al., 2006; Barends et al., 2009; Ryu et al., 2010). The sequences of these mutants are analyzed to elucidate the underlying causes of lower dark activity and higher photoactivation ratios.

Engineering a Second NIR Light-Activated Adenylyl Cyclase (IlaC=RlaC):

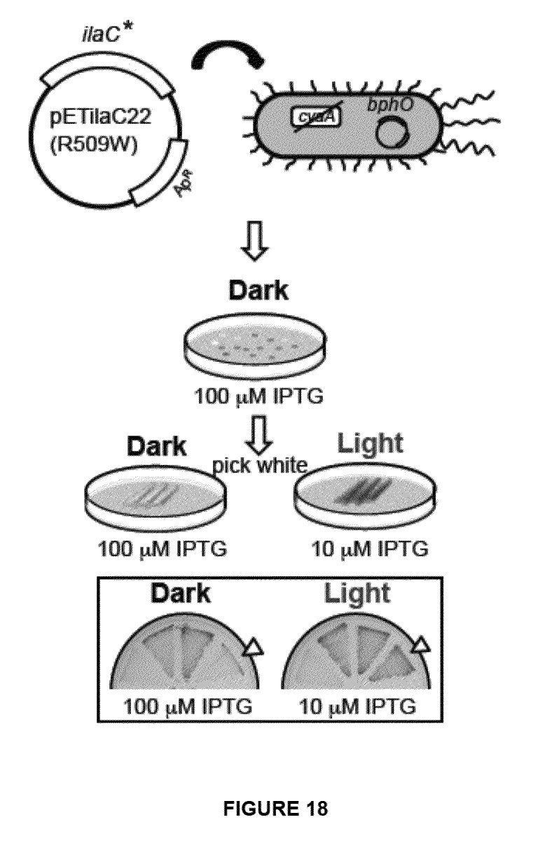

We constructed a near-infrared light activated adenylyl cyclase, IlaC, which can control cAMP-dependent processes in live animals. FIG. 18 shows the selection strategy used for isolating IlaC22 R509W mutants with decreased dark AC activities. A library of mutant IlaC22 R509W genes was generated by error-prone PCR (mutation frequency of 3-4 mutations per gene) and cloned in plasmid pET-ilaC22 R509W to replace the IlaC22 R509W gene. E. coli BL21 [DE3] cya (pT7-ho1-1) transformed with the library of mutants was grown at 30° C., in the dark, on LB plates supplemented ampicillin (100 μg/mL), kanamycin (50 μg/mL) and IPTG (100 μM). Colonies containing original pET-ilaC22 R509W produce blue colonies under these conditions. White colonies were picked and subsequently retested in the presence or absence of light at low (10 μM) and high (100 μM) IPTG concentrations. Irradiation was provided to by All-red LED Grow Light panel 225 (30.5×30.5 cm, LED Wholesalers, CA). Mutants that produced white colonies in the dark at 100 μM IPTG but blue colonies in the light at 10 μM IPTG were analyzed further. This screen led to identification of the triple mutant, IlaC22 k27 (arrowhead at the bottom of the Figure).

When expressed in cholinergic neurons of a roundworm Caenorhabditis elegans, IlaC affected worm behavior in a light-dependent manner. We engineered a series of photoactivated adenylyl cyclases (AC) (FIG. 11) designated IlaC (NIRW light-activated AC) by fusing a photosensory module from the Rhodobacter sphaeroides bacteriophytochrome DGC, BphG1 (Tarutina et al., 2006; Ryu and Gomelsky, 2014), to an AC domain from the Nostoc sp. CyaB1 protein (Kanacher et al., 2002; FIG. 11), which, like all bacterial type III ACs, works as a homodimer (Sinha and Sprang, 2006). We chose to make IlaCs because cyclic AMP (cAMP) is an important second messenger involved in regulation of diverse biological processes (Sinha and Spring, 2006; Linder, 2006). The ability to manipulate cAMP levels in specific cells or tissues in live animals is of significant interest in biomedical research.

Components of the NIRW Light-Activated AC.

For the photosensory module of the NIRW light-activated AC, we chose the PASGAF-PHY module from BphG1 from R. sphaeroides, where PAS, GAF and PHY (Phytochrome) are protein domain names (Pfam database). The truncated derivative of BphG1, BphG, where the PAS-GAF-PHY module is linked to a GGDEF domain, functions as a light-activated DGC (Tarutina et al., 2006) (FIG. 11). BphG was particularly attractive because (i) absorption maxima of its dark (Pr) and lit (Pfr) forms, 712 and 756 nm, respectively, lie within the NIRW, and (ii) its DGC activity is activated by light by approximately 11-fold, the largest photodynamic range (fold-activation) among bacteriophytochromes for which such ratio has been quantified (Ryu and Gomelsky, 2014).

For the output AC domain, we looked for a protein (i) whose AC activity is confined to the AC domains, i.e., where regulatory domains are not required for basal activity, and (ii) whose AC activity in the dark can be detected in a bacterial screening system.

CyaB1 from Nostoc sp. fit these requirements (Kanacher et al. 2002). The native CyaB1 protein has the following domain architecture, GAFGAF-PAS-PAS-AC (where AC domain is responsible for AC activity (FIG. 11). The C-terminal AC domain of CyaB1 untangled from the regulatory domains possesses some AC activity (Kanacher et al., 2002).

To monitor cAMP synthesis, we used Escherichia coli BL21[DE3] cya which lacks the endogenous AC, Cya. In this strain, expression of the chromosomal lacZ gene encoding β-galactosidase is low because of the absence of activation by the cAMP-responsive protein, CRP, also known as catabolite activator protein (Busby and Ebright, 1999). BL21[DE3] cya produces white colonies on agar containing 5-bromo-4-chloro-3-indolyl-β-Dgalactopyranoside (X-Gal) (Ryu and Gomelsky, 2014). The C-terminal AC domain of CyaB1 (amino acids [aa2]585-857) restores lacZ expression thus generating blue colonies. To endow this bacterial system with the ability to synthesize biliverdin IXα, we introduced the cyanobacterial heme oxygenase gene ho1 (Gambetta and Lagarias 2001), whose product converts heme synthesized by E. coli into biliverdin IXα.

Engineering a NIRW Light-Activated AC.

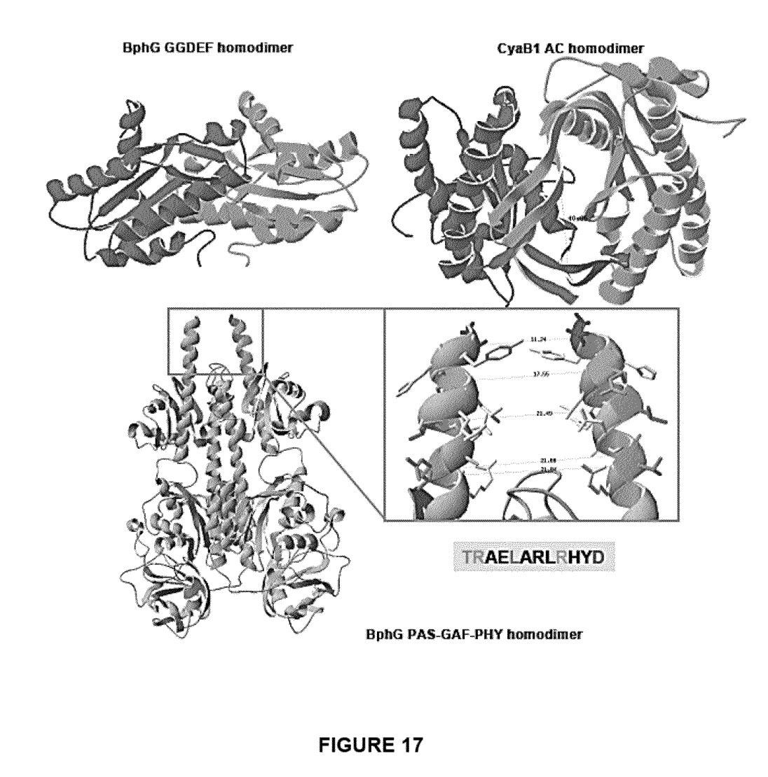

We synthesized the DNA fragment encoding the AC domain of CyaB1 (aa 585-857) and fused it to aa 526 in the unstructured (loop) region of the GGDEF domain of BphG. Leu585 of CyaB1 is also in the loop region, 8 aa upstream of the first structural element, β-strand, of the AC domain (FIG. 11 and FIG. 17).

FIG. 17 shows structural models of BphG and the AC domain of CyaB1.

Protein modeling was performed by J. Siltberg-Liberies (Ryu et al., 2014). The parallel dimer of the phytochrome domains was constructed using two PDB structures, combined in several steps. First, a parallel dimer was built using 3C2W (Yang et al., 2008) as a template. Second, a monomer based using 4GW9 (Bellini and Papiz, 2012) as a template was built in order to model the extended C-terminal α-helix from the PHY domain. In order to extend the aligned region of the C-terminal α-helix so that it could be modeled, the sequence was anchored to 4GW9 by including the anchoring sequence fragment ‘YEQFSSQVHASMQPVLITDAEGRIL’ from 4GW9. This allowed us to model the region that is critical for the estimating the distance between the extended α-helices that lead to the output domains despite low sequence identity. Third, the extended monomers were trimmed of the anchoring segment and superimposed onto the parallel dimer. Finally, the parallel dimer was modeled based on the combined template from the first three steps. This multistep modeling procedure was required to model the region important for the estimating the distance between the extended α-helices that connect PHY and AC domains. Steps 1, 3, and 4 were modeled using Swiss Model project mode, while the second step was built using the beta version of the next Swiss Model (Arnold et al., 2006). The dimer of GGDEF was constructed using 4H54 (Zahringer et al., 2013) as a template. The dimer of AC domains was constructed using 3R5G (Topal et al., 2012) as a template. Both dimers of the output domains were built using the beta version of Swiss Model (Bordoli et al., 2006).

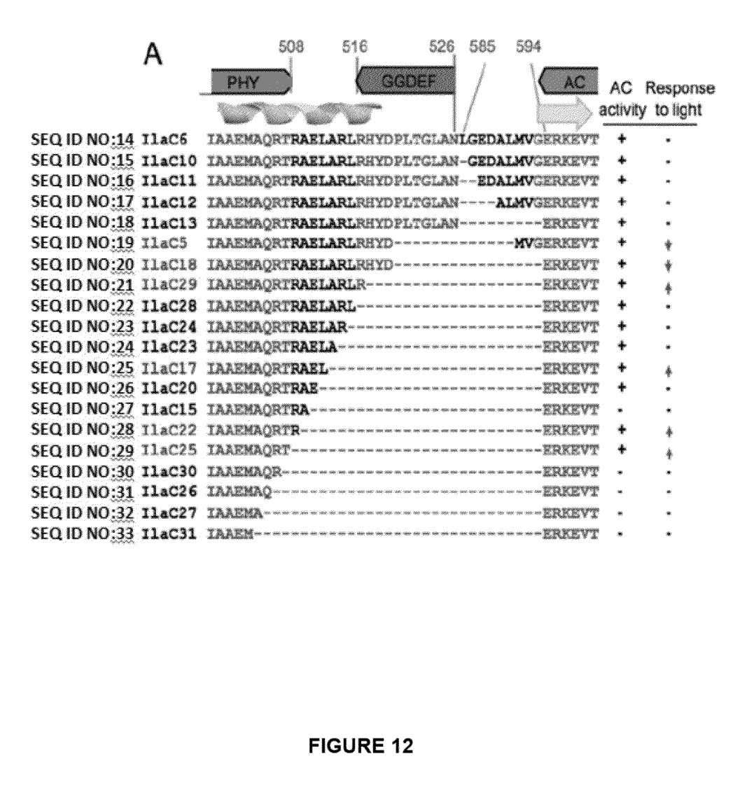

The unstructured linkers were meant to prevent potential steric interference between the fusion partners. In accord with this intent, the chimeric protein, IlaC6, namely IAAEMAQRTRAELARLRHYDPLTGILANLGEDALMVGERKEVT [SEQ ID NO:14], possessed AC activity, yet this activity was nonresponsive to light (FIG. 12 and FIG. 17). Next, we determined the minimal AC domain size that retained enzymatic activity in the BphG-CyaB1 fusions.

We fixed the fusion point in BphG at aa 526 and progressively shortened the AC domain, from Leu585 to Glu594 of CyaB1, where Glu594 is in the predicted β-strand of the AC domain (FIG. 12). All fusions in this series, IlaC 10-13, proved to be enzymatically active and nonresponsive to light (FIG. 12). The sequences of the IlaC 10-13 fusions are provided below:

IlaC10:
[SEQ ID NO: 15]
IAAEMACRTRAELARLRHYDPLTGILANGEDALMVGERKEVT
IlaC11:
[SEQ ID NO: 16]
IAAEMAQRTRAELARLRHYDPLTGILANEDALMVGERKEVT
IlaC12:
[SEQ ID NO: 17]
IAAEMAQRTRAELARLRHYDPLTGILANALMVGERKEVT
IlaC13:
[SEQ ID NO: 18]
IAAEMAQRTRAELARLRHYDPLTGILANERKEVT

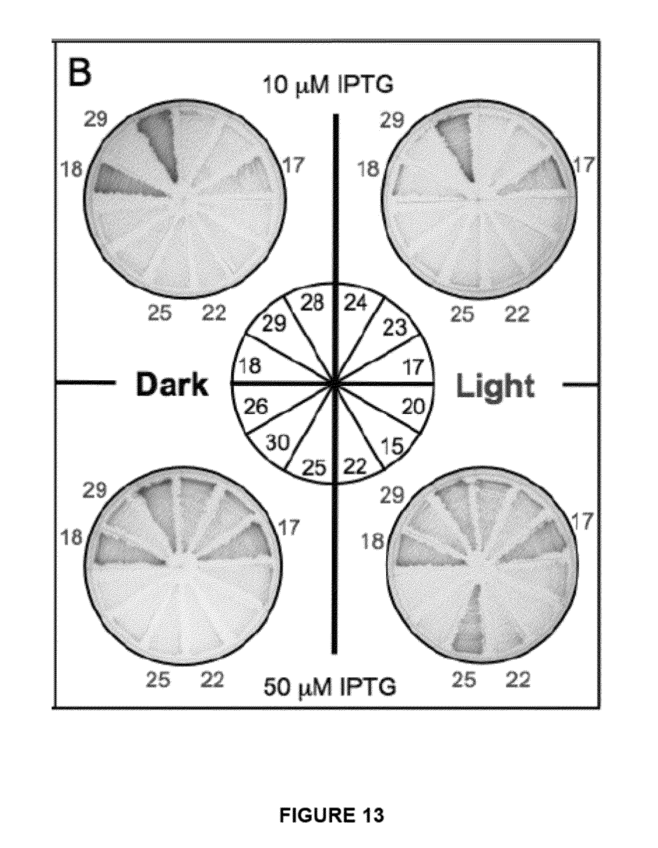

In the next round of engineering, the AC domain border was fixed at Glu594 but the unstructured region of the GGDEF domain and the α-helical linker extending from the PHY domain were subject to shortening. The fusion to Arg507 of BphG (IlaC30) IAAEMAQRERKEVT [SEQ ID NO:30] and fusions containing shorter BphG fragments (IlaC 26, 27, 31) had no AC activity (FIGS. 12 and 13) likely because of the steric hindrance between the fusion components (FIG. 11), thus the engineering space was limited to aa 508-526 of BphG. The sequences of the IlaC 26, 27 and 31 fusions are provided below:

IlaC26:
[SEQ ID NO: 31]
IAAEMAQERKEVT
IlaC27:
[SEQ ID NO: 32]
IAAEMAERKEVT
IlaC31:
[SEQ ID NO: 33]
IAAEMERKEVT.

IlaC15: IAAEMAQRTRAERKEVT [SEQ ID NO:27] was also inactive

One of the fusions in this region, IlaC18 produced a protein whose AC activity was higher in the dark than in the light, i.e., light-inactivated AC (FIGS. 12 and 13). This result showed that light induces conformational changes that are sufficient to misalign the enzymatically productive AC homodimer. The sequence of IlaC18 is provided below:

lIaC18: IAAEMAQRTRAELARLRHYDERKEVT [SEQ ID NO:20].

Notably, light-inactivated fusions were also obtained at other fusion points, e.g., IlaC5 (FIG. 11). The sequence of IlaC5 is provided below:

IlaC5: IAAEMAQRTRAELARLRHYDMVGERKEVT [SEQ ID NO:19].

Four fusions, IlaC29 (Arg516), IlaC17 (Leu512), IlaC22 (Arg509), IlaC25 (Thr508) produced the desired, photoactivated enzymes. The AC activity of these fusions differed from each other as judged by colony color on X-Gal agar at two different isopropyl-1-thio-β-D-galactopyranoside (IPTG2) concentrations used to regulate IlaC expression levels (FIG. 13). The sequences of IlaC29, IlaC17, IlaC22 and IlaC25 are set forth below:

IlaC29:
[SEQ ID NO: 21]
IAAEMAQRTRAELARLRERKEVT.
IlaC17:
[SEQ ID NO: 25]
IAAEMAQRTRAELERKEVT.
IlaC22:
[SEQ ID NO: 28]
IAAEMAQRTRERKEVT.
IlaC25:
[SEQ ID NO: 29]
IAAEMAQRTERKEVT.

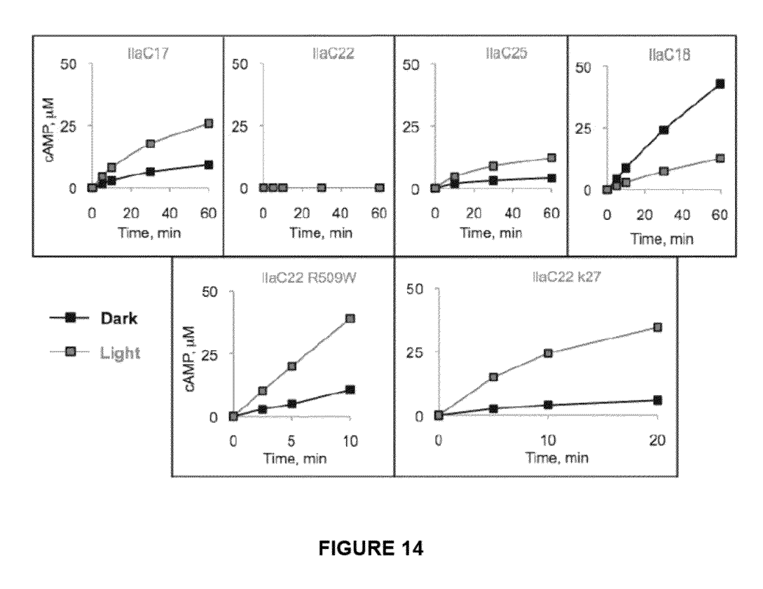

Three photoactivated cyclases, IlaC17, IlaC22 and IlaC25, as well as one photoinactivated cyclase, IlaC18, were overexpressed as C-terminal His6-fusions and purified. The AC activity of IlaC18 was decreased by 3-fold upon irradiation with 700-nm light (FIG. 14), whereas the activities of IlaC17 and IlaC25 were increased by light by approximately 2-fold (FIG. 14). In agreement with the colony phenotypes, the basal AC activity of IlaC17 was higher than the activities of IIlaC25 or IlaC22. The AC activity of IlaC22 in vitro was below detection (FIG. 14).

IlaC28, IlaC24, IlaC23 and IlaC20 were also active. Their sequences are set forth below:

IlaC28:
[SEQ ID NO: 22]
IAAEMAQRTRAELARLERKEVT.
IlaC24:
[SEQ ID NO: 23]
IAAEMAQRTRAELARERKEVT.
IlaC23:
[SEQ ID NO: 24]
IAAEMAQRTRAELAERKEVT.
IlaC20:
[SEQ ID NO: 26]
IAAEMAQRTRAEERKEVT.

Improving Photodynamic Range of the Photoactivated ACs.

Since the photodynamic range of the first-generation NIRW light-activated ACs were significantly lower than the photodynamic range of BphG, we intended to improve this parameter by random PCR-based mutagenesis. Here, we focused on IlaC22 as a template because of its low dark activity. In the light, IlaC22 produced blue colonies only when expressed at high, 50 μM, IPTG concentration. We screened the library of the PCR-mutagenized IlaC22 gene for blue colony appearance at low, 10 μM, IPTG concentration. After screening approximately 10⁵ mutant clones, we found mutants with significantly increased AC activity. The best one had an R509W (BphG numbering) substitution, right at the junction between the PHY and AC domains (FIG. 12).

The photodynamic range of AC activity of the purified IlaC22 R509W was approximately 4-fold (FIG. 14). To further improve the photodynamic range, we mutagenized the ilaC22 R509W gene at high mutation frequency. Our primary goal was to decrease the dark AC activity of IlaC22 R509W. We therefore searched for white colonies at high, 100 μM, IPTG, in the dark. The strategy used in this screen is illustrated in FIG. 18. After screening of >10⁵ mutant clones, we identified a derivative, IlaC22 k27, that had significantly lower activity in the dark but only slightly lower activity in the light, compared to IlaC22 R509W (FIG. 14), which resulted in the photodynamic range of 6-fold. IlaC22 k27 was found to contain three new mutations, compared to IlaC22 R509W, two of which were in the BphG photosensory module (L164M, Q371H) and one in the AC domain (Q670R of CyaB1).

The protein sequences of IlaC22 k27 are provided below. Four mutations distinguishing IlaC22 k27 from IlaC22 are shown in boldface in normal font (R209W) and boldface larger font. Y259 mutated in and IlaC22 k27 Y259F is show in in boldface and smaller font. The sequence derived from BphG is shown in brown; the sequence derived from CyaB1 is shown in italics, the C-terminal His6-tag at the end of the sequence is shown in black normal size font.

[SEQ ID NO: 34]
MARGCLMTISGGTFDPSICEMEPIATPGAIQPHGALMTARADSGRVAHASVNLG
EILGLPAASVLGAPIGEVIGRVNEILLREARRSGSETPETIGSFRRSDGQLLHLHAFQSGDY
MCLDIEPVRDEDGRLPPGARQSVIETFSSAMTQVELCELAVHGLQLVMGYDRVMAYRFG
ADGHGEVIAERRRQDLEPYLGLHYPASDIPQIARALYLRQRVGAIADACYRPVPLLGHPEL
DDGKPLDLTHSSLRSVSPVHLDYMQNMNTAASLTIGLADGDRLWGMLVCHNTTPRIAGP
EWRWGMIGQVVSLLLSRLGEVENAAETLARQSTLSTLVERLSTGDTLAAAFVAADQLIL
DLVGASAAVVRLAGHELHFGRTPPVDAMQKVLDSLGRPSPLEVLSLDDVTLRHPELPELL
AAGSGILLLPLTSGDGDLIAWFRPEHVQTITWGGNPAEHGTWNPATQRMRPRASFDAWK
ETVTGRSLPWTSAERNCARELGEAIAAEMAQRT [symbol] ERKEVTVLFSDIRGYTTLTENLGAAE
VVSLLNQYFETMVEAVFNYEGTLDKFIGDALMAVFGAPLPLTENHAWQAV [symbol] SALDMRQRL
KEFNQRRIIQAQPQIKIGIGISSGEVVSGNIGSHKRMDYTVIGDGVNLSSRLETVTKEYGCDI
ILSEFTYQLCSDRIRVRQLDKIRVKGKHQAVNIYELISDRSTPLDDNTOEFLFHYHNGRTAY
LVRDFTQAIACFNSAKHIRPTDQAVNIHLERAYNYQQTPPPPQWDGVWTIFTKHHHHHH.

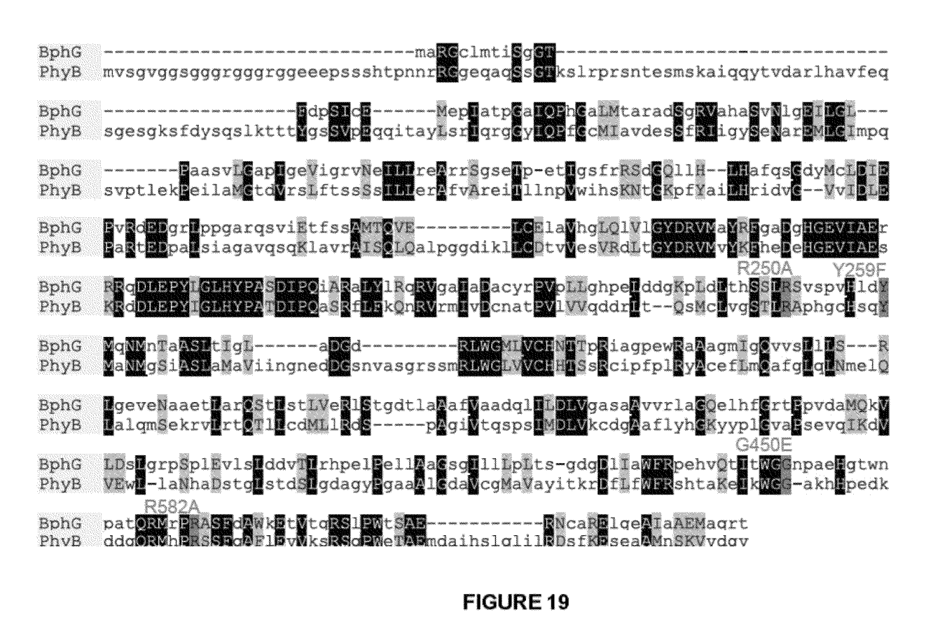

FIG. 19 illustrates identification of residues involved in slowing the dark thermal recovery of IlaC. Shown is alignment of the R. sphaeroides BphG bacteriophytochrome (SEQ. ID NO:34) and the Arabidopsis thaliana phytochrome PhyB. Residues of PhyB affecting its photocycle and conserved in BphG are highlighted in dark gray. Mutations of PhyB that extend the lit (Pfr) state of PhyB are shown in light gray. The Arabidopsis thaliana PhyB amino acid sequence is publicly available on Genbank, identified as NCBI Reference Sequence: NM_{—}127435.3.

Since the photodynamic range of IlaC22 k27 was within 2-fold of the photodynamic range of the BphG, we did not attempt to increase it further. Instead, we focused on modifying another important parameter, i.e., stability of the photoactivated state.

Extending Lifetime of the Light-Activated State of an AC.

Following photoactivation, bacteriophytochromes in the lit (Pfr) state spontaneously return to the ground, dark (Pr), state via thermal reversion (Rockwell and Lagarias, 2006). The half-life of IlaC22 k27 in the Pfr state is 46±3 s (FIG. 14A). A relatively short half-life is desirable for optogenetic applications that require short pulses of cAMP, whereas applications involving sustained light-induced increases in cAMP levels will benefit from enzymes with more stable lit state.

To increase lifetime of the lit state of IlaC22 k27, we relied on success in extending this parameter in the Arabidopsis thaliana phytochrome PhyB (Adam et al., 2011; Zhang et al., 2013). Four residues in the proximity of the chromophore involved in controlling dark recovery of PhyB are conserved in BphG (FIG. 19). We introduced in IlaC22 k27 the same mutations as those that prolonged the half-life of PhyB.

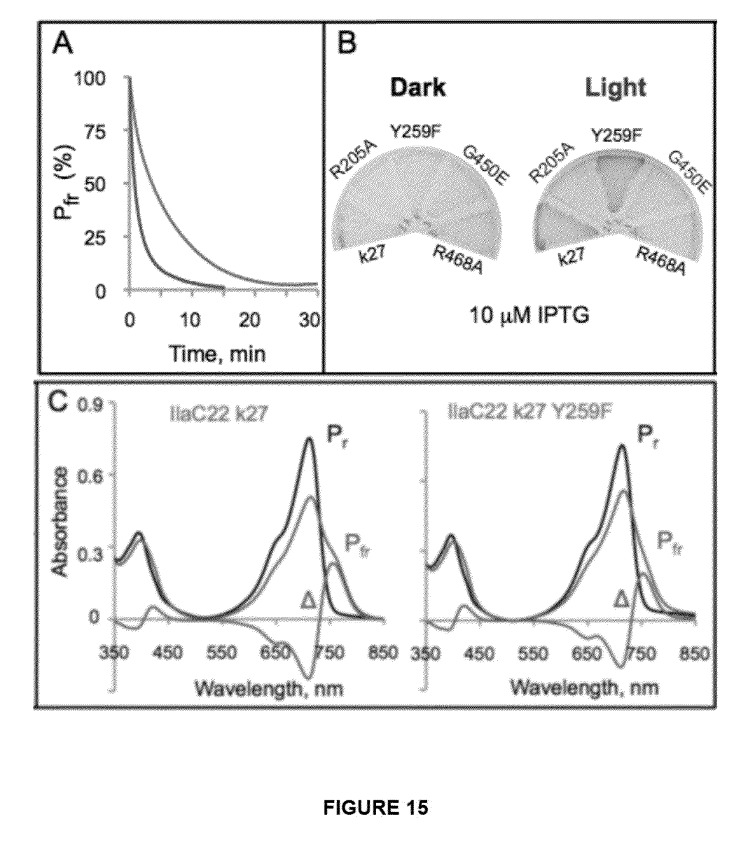

Three mutations (R205A, G450E, and R468A) resulted in the loss of AC activity and were discarded (FIG. 15B). The fourth mutant, IlaC22 k27 Y259F, was purified and characterized in vitro. Its absorption maxima were slightly shifted compared to IlaC22 k27, i.e., Pr (713 nm) and Pfr (755 nm) (FIG. 15C), and the half-life of thermal reversion was significantly (by 4.3-fold), increased to 197±9 s, compared to IlaC22 k27 (FIG. 15A).

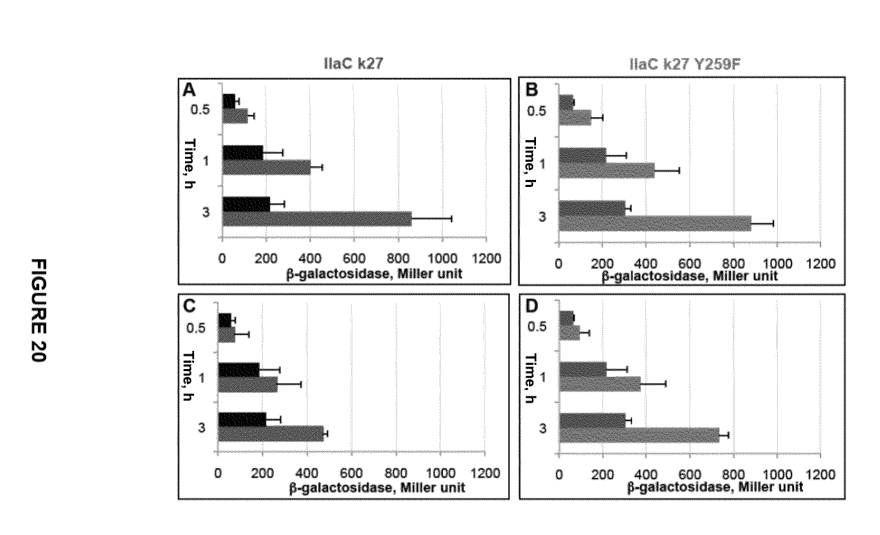

The higher stability of the lit state of IlaC22 k27 Y259F was expected to allow its use in the pulsed light regiments, which may decrease such negative effects of constant irradiation as tissue heating. To test this possibility, we compared the effect of IlaC22 k27 and IlaC22 k27 Y259F on the cAMP-CRP-dependent lacZ gene expression in E. co/i (FIG. 20).

FIG. 20 shows effects of IlaC k27 and IlaC k27 Y259F on cAMP-CRP-dependent lacZ gene expression in E. coli. Strain BL21[DE3] cya (pT7-ho1-1) containing either pET-ilaCK27 or pET-ilaC k27 Y259F were grown in culture tubes on a shaking platform at 30° C. in LB supplemented with ampicillin (50 μg/mL) and kanamycin (50 μg/mL). When optical densities of the cultures reached A₆₀₀ 1.5, IPTG (1 mM, final concentration) was added to induce IlaC gene expression. Irradiation was provided to by All-red LED Grow Light panel. Tubes containing “dark” samples were wrapped in aluminum foil to avoid light exposure. Samples were withdrawn at the indicated time points (0.5, 1 and 3 h) post-induction for determination of β-galactosidase activities, which was measured by the Miller method (Sambrook et al., 1989). (A), IlaC k27; (B), IlaC k27 Y259F. A, B, constant irradiation; C, D, pulsed irradiation (30 s light, 90 s dark). Black or dark-grey bars, dark; light gray bars, light. Error bars show standard deviation derived from three independent experiments. The β-galactosidase measurements were performed by A. I. Lyuksyutova (Ryu et al., 2014).

While both ACs showed similar increases in lacZ expression in constant light, compared to the dark, when pulsed light (30 s light, 90 s dark) was used, the IlaC22 k27 Y259F mutant outperformed IlaC22 k27 (FIG. 20), as expected.

### Example 2

### Engineering a Near-Infrared Light-Activated Executioner Caspase

Executioner caspases are terminal apoptosis-inducing proteases. They catalyze cleavage of essential cellular proteins thus irreversibly leading to apoptosis (reviewed in Crawford & Wells, 2011). Photoactivated executioner caspases can thus be used to induce specific spatiotemporal apoptosis to study molecular and cellular topics in animal development or disease. Caspase-3 functions as homodimer (reviewed in Mackenzie & Clay, 2008). In order to gain proteolytic activity, procaspase-3 undergoes proteolytic activation carried out by the upstream initiator caspases. However, Clark et al. constructed a noncleavable mutant D9A D28A D175A (designated D3A). An additional mutation, V266E, makes procaspase-3 active without proteolytic processing. The V266E mutant protein has a 60-fold higher enzymatic activity compared to the procaspase-3 D3A (which is inactive), and approximately ⅓ of the activity of the fully processed (active) caspase-3 (Pop et al., 2003; Walters et al., 2009). Since the procaspase-3 D3A V266E homodimer is intrinsically active, a distorted homodimer interface in the dark can be engineered, and restored by the light-induced helix rotation. This is conceptually identical to the task of engineering NIR light activated adenylyl cyclase.

The screening system developed by Hayashi et al. (Hayashi et al., 2009) for high throughput screening of DNA libraries in Saccharomyces cerevisiae is used for screening of photoactivated procaspase-3 fusions. In this system, the (pro)caspase-3 activity is monitored in yeast using a blue/white colony screening based on the level of expression of the lacZ reporter. Expression of the lacZ reporter gene is dependent on the transcription activator whose cellular localization is determined by the caspase-3 activity. If caspase-3 is active, the transcription activator is cleaved off from its transmembrane domain, released from the membrane, moves to the nucleus and activates lacZ expression. If caspase activity is low, the transcription activator remains as a fusion with the transmembrane domain and therefore is sequestered to the membrane and unable to activate lacZ expression. Active caspase-3 releases the transcription activator by cleaving at its recognition site, DEVD, engineered between the transmembrane and activator modules of the transcription activator. In addition to lacZ, LEU2 (providing for leucine prototrophy when expressed in the S. cerevisiae LEU2 mutant) can also be used as a reporter of caspase-3 activity.

The procaspase-3 D3A V266E is fused to the photoreceptor PAS-GAF-PHY module of BphG and expressed in S. cerevisiae under the galactose-inducible GAL1 promoter, in a dose-dependent fashion with the varying galactose concentration in the media. The photoactivated caspase-3 derivatives are identified as blue color colonies on X-gal leucine-deficient media on plates grow in the light. Responsiveness of such colonies to light is subsequently investigated upon comparing colony color in the light and in the dark as shown for photoactivated adenylyl cyclase (FIG. 7). The yeast strain also expresses the R. sphaeroides heme oxygenase BphO1 that provides biliverdin to the chimeric caspase.

First-generation photoactivated caspases identified are subjected to iterative mutagenesis and screening to identify those with the lowest dark activities and the highest photoactivation ratios (similar to those described for the photoactivated adenylyl cyclase). The optimized photoactivated procaspase-3 D3A V266E proteins are purified via the C-terminal His6-tag (Pop et al., 2003), and assayed in vitro using commercially available fluorescence- or chromogenic assays of caspase-3 activity.

### Example 3

### NIRW Light-Activated Control of Caenorhabditis elegans Behavior

To test performance of the NIRW light-activated AC oin an animal model, we expressed IlaC k27 in cholinergic neurons of the roundworm C. elegans.

### Materials and Methods

Microbiological Methods.

Escherichia coli BL21[DE3] cya lacking endogenous adenylyl cylase CyaA (26) and containing two plasmids, pT7-ho1-1 (25) that express the Synechocystis sp. heme oxygenase ho1 (Gambetta & Lagarias, 2001) and pETilaC# (expressing IlaC proteins) were used for IlaC screening. Strains were grown at 30° C. in LB supplemented with X-gal (40 μg/mL), ampicillin (50 μg/mL) and kanamycin (25 μg/mL). IPTG was added for induction of IlaC proteins. For light-sensitive experiments, Petri dishes were placed onto All-red (660 nm) LED2 Grow Light panels 225 (30.5×30.5 cm, LED Wholesalers, CA). Light irradiance was approximately 0.2 mW cm⁻². For growth in the dark, Petri dishes were wrapped in aluminum foil.

Recombinant DNA Techniques.

The DNA fragment of bphG 1 gene (RSP4191) encoding the photosensory PAS-GAF-PHY module was amplified by PCR from the R. sphaeroides 2.4.1 genome. The DNA fragment of cyaB1 from Nostoc (formerly Anabaena) sp. PCC 7120 was synthesized by BioBasics, Inc. with the codon usage optimized for R. sphaeroides. Two fragments were joined by fusion PCR using GoTaq (Promega) and subsequently cloned into the XbaI and HindIII sites of pET23a(+) (Invitrogen) to yield a series of plasmids, pETilaC#. Each of these plasmids encodes a unique BphGCyaB1::His6 fusion protein. Site-directed mutagenesis was performed using QuickChange kit (Stratagene). Error-prone PCR mutagenesis was carried out using GeneMorph II Random mutagenesis kit (Agilent Technologies).

The Punc-17:ilaC plasmid, pNQ149, was constructed using the MultiSite Gateway Three-Fragment Vector Construction Kit (Invitrogen). The ilaC22 k27 cDNA PCR product was first cloned into the pdonr221 entry vector by BP cloning. pNQ149 was then constructed by using the LR reaction to combine the unc-17 promoter (obtained from the Promoeome library in the pdonrP4-P1r entry vector), the ilaC22 k27 cDNA in the pdonr221 entry vector, and the unc-54 3′UTR in the pdonrP2R-P3 entry vector.

Protein Purification and AC Assays.

The IlaC proteins were purified as C-terminal His6-tagged fusions using Ni-NTA affinity chromatography (Novagen). AC assays were performed using freshly purified proteins at room temperature, essentially as described earlier (Ryjenkov et al. 2005).

Spectroscopy.

Electronic absorption spectra were recorded with a UV-1601 PC UV-visible spectrophotometer (Shimadzu) at room temperature. Protein solution (100 μL) in a 10-mm light path quartz cuvette was irradiated by 1-W (700 nm) LED directly in the spectrophotometer from the top of the cuvette.

Bioinformatics.

Multiple sequence alignments were generated using MUSCLE (Edgar RC 2004). Secondary structures predictions were performed using Jpred3 server (Bordoli and Schwede, 2006). Modeling of 3D structures was done using Swiss Model (Barber and Barton 2008) project mode and beta version of the next Swiss Model (Benkert and Schwede 2011).

Protein Overexpression and Purification.

The IlaC proteins were purified as C-terminal His6-tagged fusions using Ni-NTA affinity chromatography according to specifications of the manufacturer (Novagen). Protein purification was performed under green light. The overnight cultures of E. coli BL21[DE3] expressing the IlaC::His6 proteins were grown in LB supplemented with appropriate antibiotics at room temperature to A600 0.7. Protein expression was induced with IPTG at final concentration of 0.5 mM, and the cultures were incubated with shaking at 250 rpm at 18° C. for additional 20 h. Bacteria were collected by centrifugation at 4,000×g for 10 min, washed and resuspended in the binding buffer (50 mM sodium phosphate [pH 8.0], 300 mM NaCl) supplemented with 0.2 mM phenylmethylsulfonyl fluoride and 10 mM imidazole. Cells were disrupted using a French pressure cell, and cell debris was removed by centrifugation at 35,000×g for 45 min at 4′C. Three milliliters (bed volume) of Ni-NTA resin (Novagen) preequilibrated with the binding buffer were added to the soluble cell extract derived from a 1 L culture and agitated for 1 h at 4° C. The mix was loaded onto a column, and the resin was washed with 200 mL of column binding buffer. Fractions were eluted with 12 mL of binding buffer containing 250 mM imidazole. The proteins were either used immediately or stored at −80° C. in 20% v/v glycerol (final concentration). Protein concentrations were measured using aBradford protein assay kit (BioRad) with bovine serum albumin as the protein standard. Proteins were analyzed using SDS-PAGE.

AC Assays.

A standard reaction mixture (300 μl) contained 5 mM enzyme in the assay buffer (50 mM Tris-HCl [pH 8.0], 10% glycerol, 10 mM MgCl2, 0.5 mM EDTA). The protein was irradiated either with dim green light or far-red light emitted from a 1-W (700 nm) LED at the approximate irradiance of 0.2 mW cm-2. The reaction was started by the addition of ATP.

Aliquots (50 μL) were withdrawn at different time points and immediately boiled for 5 min. The precipitated protein was removed by centrifugation at 15,000×g for 5 min. The supernatant was filtered through a 0.22-μm pore size filter (MicroSolv, NJ). cAMP levels were analyzed by reversed-phase high-pressure liquid chromatography (HPLC) as described earlier (Ryjenkov et al. 2005).

C. elegans Cultivation and Transgenesis.

Animals were cultivated on nematode growth medium (NGM) agar and were fed E. coli DA837 derived from strain OP50 (Davis et al. 1995). All experiments were performed onhermaphrodites. The following strains were used in this study: N2 (Bristol), NQ719 qnEx386[Punc-17:iLac; Pmyo-2:mCherry], NQ721 qnEx388[Punc-17:iLac; Pmyo-2:mCherry]. Transgenic animals were created by microinjection (Stinchcomb et al. 1985) using a Leica DMIRB inverted DIC microscope equipped with an Eppendorf Femtojet microinjection system. N2 animals were injected with 25 ng/μL of pNQ149 in combination with 5 ng/μL of pCFJ90 (Pmyo-2:mCherry) and 120 ng/μL 1 kb molecular weight ladder (New England Biolabs).

C. elegans Behavioral Assays.

Animals were grown in the dark for one generation on NGM agar seeded with DA837 bacteria. For the assays performed on agar, L4 larvae were transferred to NGM plates seeded withDA837 and grown to early adulthood overnight. Individual adult animals were transferred in the presence of green light onto an NGM plate without bacteria and left unperturbed for 15 min. Animals were observed using a Leica MZ16 stereomicroscope. During this time the animals were exposed to green light for 30 s, red light for 30 s, and green light for 30 s. Body bends were counted by the observer. Irradiation was provided by LED Color Changing Kit IP66 (LED Wholesalers, CA). No biliverdin IXα was added to the agar.

For the swimming assays, we followed, with minor modifications, the protocols described by Weissenberger et al. (2011). L4 larvae were transferred to NGM plates seeded with DA837 bacteria supplemented with 1 mM biliverdin hydrochloride (Sigma) and grown to early adulthood for one day.

Individual early adult animals were transferred in the presence of green light into a 10 μL drop of NGM and M9 in a 1:1 ratio supplemented with 1 mM biliverdin hydrochloride. Animals were video monitored for 2 minusing a USB 2.0 monochrome camera (ImagingSource, model DMK 72AUC02), mounted on a Leica MZ16 stereomicroscope. During the 2 min recording, the animals were exposed to green light for 30 s, red light for 30 s, and green light again for 60 s. Body bend frequency was counted by observing the video recordings.

Previously, a blue-light activated AC, PACα, has been utilized in C. elegans as a tool for optogenetic manipulation of behavior. Expression of PACα in the cholinergic neurons, using the promoter for the vesicular acetylcholine transporter unc-17, followed by stimulation with blue light resulted in increased locomotory activity (Weissberger et al., 2011). However, blue light activates a C. elegans avoidance response and is toxic upon prolonged irradiation (Weissberger et al., 2011; Edwards et al. 2008), thus confounding the interpretation of the behavioral response to cAMP optogenetic manipulation. Therefore, we used IlaC for behavioral analyses in C. elegans.

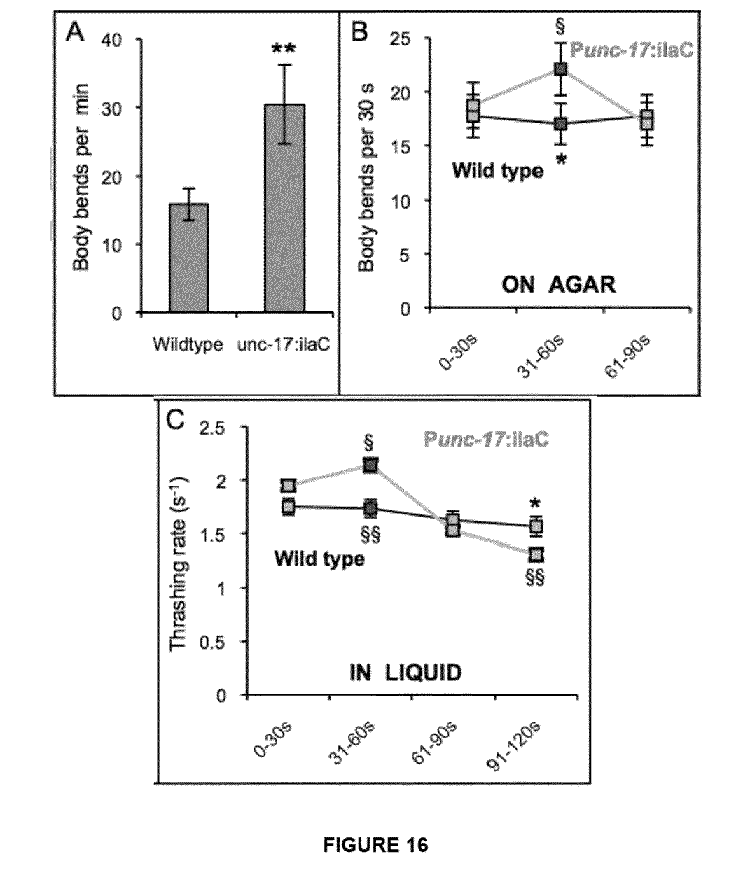

We generated transgenic animals expressing IlaC22 k27 in cholinergic neurons using the unc-17 promoter. IlaC22 k27 transgenic animals cultivated on an agar surface and exposed to daily light from the environment were more active than wild-type animals, as evident by the frequency of their body bends (FIG. 16A).

Hyperactivity is characteristic of animals with increased activity of cAMP-dependent protein kinase A (PKA), such as mutants for the gene kin-2, which encodes for the regulatory subunit of PKA (Schade et al., 2005). To test the effect of red light, we grew wild-type and Punc-17:ilaC animals in the dark for a single generation.

Individual animals were transferred to an agar surface that did not contain food. Animals were monitored under monochromatic LED irradiation for 90 s. During this time, body bends were counted during the following light regiment: green light 0-30 s, red 31-60 s, and green 61-90 s. While control animals did not alter their locomotory activity in response to red light (FIG. 16B), Punc-17:ilaC animals performed more body bends in the presence of red light (FIG. 16B).

We also monitored the effects of AC activation in cholinergic neurons on the frequency of thrashing movements in liquid medium. Swimming animals, which were reared in the dark, were video monitored for a total of 2 min during the following light regiment: green 0-30 s, red 31-60 s, green 61-120 s. While control animals did not alter their thrashing rate in response to red light, Punc-17:ilaC animals increased their thrashing rates when exposed to red light (FIG. 16C). Interestingly, their thrashing rates significantly decreased during the second exposure to green light (FIG. 16C), due to post-stimulatory fatigue. Thus, our results indicate that the NIRW light-activated AC can be used as a tool for optogenetic manipulation of cAMP levels in animals

Discussion:

Unique photochemical properties of bacteriophytochromes, i.e., (i) light absorbance in the NIRW, optimal for use in red-blooded animals; (ii) naturally available in animal cells chromophore (biliverdin IXα), and (iii) innocuous nature of NIRW light, position them as superior photoreceptors for optogenetic applications in animals (Sambrook et al., 1989). However, bacteriophytochrome engineering for optogenetic applications has been hindered by the lack of understanding of mechanisms through which light-induced conformational changes induce changes in output domains. Two most common bacteriophytochrome types, histidine kinases and DGCs, operate as homodimeric enzymes, where proper alignment of two monomeric kinase or GGDEF domains is essential for enzymatic activity. The constructed-here NIRW light-activated ACs, IlaCs showed that α-helices extending from the PAS-GAF-PHY photosensory modules are primarily responsible for alignment of the output domains (FIG. 11) and that the light-induced movements mediated by the α-helices can regulate alignment of unrelated, heterologous domains whose activity requires properly aligned homodimers. The recently solved structures of the photosensory module of the Deinoccoccus bacteriophytochrome showed that the distance between the signaling α-helices changes by as much as 30 A in response to light (Takala et al., 2014).

We have enabled the following for future homodimeric bacteriophytochrome engineering: First, we have provided guidance for the choice of the output domains and screening schemes. For example, the ability of the AC domains of CyaB1 to spontaneously homodimerize helped us to distinguish between enzymatically active and inactive fusions (FIGS. 12 and 13). The high sensitivity of the screening system employed here was useful for detecting relatively low (2-fold) photodynamic ranges (FIG. 13). An engineering strategy that involves (i) construction of permissive fusions (involving unstructured linkers) as the first step, (ii) subsequent minimization of the output domain size, and finally, (iii) shortening (or extending) signaling α-helices, are also applicable.

We have also shown that photoactivation ratios of the first-generation light-responsive fusions can be significantly improved by extensive mutagenesis.

The energy associated with the light-induced conformational changes should be sufficient to manipulate the output domain alignment. For overly tight homodimers, monomer interactions can be weakened. Another important issue is reasonable correspondence in distances between fusion points in the signaling and output components. We estimated the distance between the α-helical residues of BphG that resulted in photoactivated fusions were in the range of 11-22 Å, while the distances the distances between the N-terminal β-strands of the catalytically inactive CyaB1 AC domainhomodimer were approximately 40 Å (see FIG. 17). These distances turned out to fit nicely in the 10-40 Å spread between the α-helices of the Deinococcus BphP in the dark and lit states (Takala et al., 2014).

We found not one but several photoactivated IlaC fusions. Interestingly, they differed from each other by either 3-4 residues (IlaC29=IlaC17+4 aa; IlaC25=IlaC17−4 aa; IlaC22=liaC17−3 aa) (FIG. 12), which places their AC domains roughly in the same helical phase but separated by one or two α-helical turns (1 turn=3.6 aa [Lehninger et al., 2000]) (FIG. 17). This result is consistent with the model where α-helices act as rods involved in aligning rigid output domains located at their tips. It is therefore unsurprising that the 1 or 2-aa shifts out of helical phase destroy proper domain alignment and result in the loss of light responsiveness (FIG. 12).

IlaC expressed in neurons of the roundworm C. elegans affected behavior in response to light. In the case of C. elegans, the main advantages of IlaC over blue-light activated ACs are the absence of the photoavoidance response and the lack of phototoxicity associated with prolonged exposure to blue light (Weissenberger et al., 2011; Edwards et al., 2008). However, IlaC will be most useful in applications in deep mammalian tissues inaccessible by blue light.

Our demonstration that homodimeric bacteriophytochromes are amenable to protein engineering and recent progress in structural understanding of dark-to-light conformational changes (Takala et al., 2014) enables the design of new NIRW light-activated proteins.

Since activities of many signal transduction components depend on homodimerization, including membrane receptors, cyclic nucleotide phosphodiesterases, certain protein phosphatases, proteases, nucleases, and transcription factors, we have enabled significant expansion of the optogenetic toolset involving NIRW light.

Optimized versions of the photoactivated chimeric enzymes, adenylyl- and guanylyl cyclases and procaspase-3 can be expressed under cell- or tissue-specific promoters and delivered to desired organisms via gene delivery procedures known in the art by those of ordinary skill in the art without undue experimentation.

This work has shown for the first time the engineering of a near-infrared light-activated heterologous activities based on bacteriophytochrome photoreceptor modules, revealed engineering principles applicable to a variety of homodimeric proteins, and demonstrated the utility of random mutagenesis and screening in test organisms for identifying bacteriophytochrome-based proteins with improved photoactivation ratios and low dark activities.

The methods provided herein have been described in terms of specific illustrations. It will be appreciated by those of ordinary skill in the art that reagents, starting materials, and process steps and conditions can be varied without undue experimentation by substitution of equivalents thereto to achieve analogous results and produce analogous fusion proteins and DNA encoding them. All such variations are considered equivalent to those specifically illustrated herein, and are intended to be covered the claims hereof.

### REFERENCES

Ád\dot{a}m É, Hussong A. Bindics J, Wüst F, Viczi\dot{a}n A, Essing M, Medzihradszky M, Kircher S, Schäfer E, Nagy F (2011) Altered dark- and photoconversion of Phytochrome B mediate extreme light sensitivity and loss of photoreversibility of the phyB-401 mutant. PLoS ONE 6, e27250
Adamantidis A R, Zhang F, Aravanis A M, Deisseroth K, de Lecea L (2007) Neural substrates of awakening probed with optogenetic control of hypocretin neurons. Nature 450, 420-4
Airan R D, Thompson K R, Fenno L E, Bernstein H, Deisseroth K (2009) Temporally precise in vivo control of intracellular signaling. Nature 458, 1025-9
Arnold K, Bordoli L, Kopp J, Schwede T (2006) The SWISS-MODEL Workspace: A web-based environment for protein structure homology modelling. Bioinformatics 22, 195-201
Auldridge M E, Forest K T (2011) Bacterial phytochromes: more than meets the light. Crit Rev Biochem Mol Biot 46, 67-88
Barends, T R M, Hartmann E, Griese J, Kirienko N V, Ryjenkov D A, Reinstein J. Shoeman R L, Gomelsky M, Schlichting I (2009) Structure and mechanism of a light-regulated cyclic nucleotide phosphodiesterase. Nature 459, 1015-8
Bellini D, Papiz M Z (2012) Structure of a bacteriophytochrome and light-stimulated protomer swapping with a gene repressor. Structure 20:1436-46
Benkert P, Biasini M, Schwede T (2011) Toward the estimation of the absolute quality of individual protein structure models. Bioinformatics 27, 343-50
Bhoo, S-H, Davis, S J, Walker, J., Karniol B, Vierstra R (2001) Bacteriophytochromes are photochromic histidine kinases using a biliverdin chromophore. Nature 414, 776-9
Bulina M E, Chudakov D M, Britanova O V, Yanushevich Y G, Staroverov D B, Chepurnykh T V, Merzlyak E M, Shkrob M A, Lukyanov S, Lukyanov K A (2006) A genetically encoded photosensitizer. Nat Biotechnol 24, 95-9
Bruder S, Linder J U, Martinez S E, Zheng N, Beavo J A, Schultz J E (2005) The cyanobacterial tandem GAF domains from the CyaB2 adenylyl cyclase signal via both cAMP-binding sites. Proc Natl Acad Sci USA 102, 3088-92
Busby S, Ebright R H (1999) Transcription activation by catabolite activator protein (CAP). J Mol Biof 293, 199-213
Byrnes K R, Waynant R W, liev I K, Wu X, Barna L, Smith K, Heckert R, Gerst H, Anders J J (2005) Light promotes regeneration and functional recovery and alters the immune response after spinal cord injury. Lasers Surg Med 36, 171-85
Cardin J A, Carlén M, Meletis K, Knoblich U, Zhang F, Deisseroth K, Tsai L H, Moore C I (2009) Driving fast-spiking cells induces gamma rhythm and controls sensory responses. Nature 459, 663-7
Cho M-H, Yoo Y, Bhoo S-H, Lee, S-W (2011) Purification and characterization of a recombinant bacteriophytochrome of Xanthomonas oryzae pathovar oryzae. Protein J 30, 124-31
Chin, K V, Yang W L, Ravatn R, Kita T, Reitman E, Vettori D, Cvijic M E, Shin M, lacono L (2002) Reinventing the wheel of cyclic AMP novel mechanisms of cAMP signaling. Ann N Y Acad Sci 968, 49-64
Cole C, Barber J D, Barton G J (2008) The Jpred 3 secondary structure prediction server. Nucl Acids Res 36. W197-W201
Crawford E D, Wells J A (2011) Caspase substrates and cellular remodeling. Annu Rev Biochemistry 80, 1055-87
Cubeddu R, Pifferi A, Taroni P, Torricelli A, Valentini G (1999) Noninvasive absorption and scattering spectroscopy of bulk diffusive media: An application to the optical characterization of human breast. Appl Phys Lett 74, 874-6
Davis M W, Somerville D, Lee R Y, Lockery S, Avery L, Fambrough D M (1995) Mutations in the Caenorhabditis elegans Na,K-ATPase alpha-subunit gene, eat-6, disrupt excitable cell function. J Neurosci 15, 8408-18
De N, Navarro M V, Raghavan R V, Sondermann H (2009) Determinants for the activation and autoinhibition of the diguanylate cyclase response regulator WspR. J Mol Biol 393, 619-33
De N, Pirruccello M, Krasteva P V, Bae N, Raghavan R V, Sondermann H (2008) Phosphorylation-independent regulation of the diguanylate cyclase WspR. PLoS Biol 6, e67
Desmet K D, Paz D A, Corry J J, Eells J T, Wong-Riley M T, Henry M M, Buchmann E V, Connelly M P, Dovi J V, Liang H L, Henshel D S, Yeager R L, Millsap D S, Lim J, Gould L J, Das R. Jett M, Hodgson B D, Margolis D, Whelan H T (2006) Clinical and experimental applications of NIR-LED photobiomodulation. Photomed Laser Surg 24, 121-8
Edgar R C (2004) MUSCLE: multiple sequence alignment with high accuracy and high throughput. Nuci Acids Res 32, 1792-7
Edwards S L, Charlie N K, Milfort M C, Brown B S, Gravlin C N, Knecht J E, Miller K G (2008) A novel molecular solution for ultraviolet light detection in Caenorhabditis elegans. PLoS Biol 6, e198
Efetova M Petereit L, Rosiewicz K, Overend G, Hauβig F, Hovemann B T, Cabrero P, Dow J A, Schwärzel M (2013) Separate roles of PKA and EPAC in renal function unraveled by the optogenetic control of cAMP levels in vivo. J Cell Sci 126, 778-788.
Fang Q, Carp S A, Selb J, Boverman G, Zhang Q, Kopans D B, Moore R H, Miller E L, Brooks D H, Boas D A (2009) Combined optical imaging and mammography of the healthy breast: optical contrast derived from breast structure and compression. IEEE Trans Med Imaging 28, 30-42
Gasser C, Taiber S, Yeh C, Wittig C H, Hegemann P, Ryu S, Wunder F, Möglich A. (2014) Engineering of a red-light-activated human cAMPlcGMP-specific phosphodesterase. Proc Natl Acad Sci USA 111, 8803-8
Gambetta G A, Lagarias J C (2001) Genetic engineering of phytochrome biosynthesis in bacteria. Proc Natl Acad Sci USA 98, 10566-71
Georgianna W E, Deiters A (2010) Reversible light switching of cell signaling by genetically encoded protein dimerization. Chembiochem 11, 301-3
Gomelsky M, Hoff W H (2011) Light helps bacteria make important lifestyle decisions. Trends Microbiol 19, 441-8.
Gradinaru V, Mogri M, Thompson K R, Henderson J M, Deisseroth K (2009) Optical deconstruction of parkinsonian neural circuitry. Science 324, 354-9
Gradinaru V, Zhang F, Ramakrishnan C, Mattis J, Prakash R, Diester I, Goshen I. Thompson K R, Deisseroth K (2010) Molecular and cellular approaches for diversifying and extending optogenetics. Cell 141, 154-65
Hockberger P E, Skimina T A, Centonze V E, Lavin C. Chu S, Dadras S, Reddy J K, White J G (1999) Activation of flavin-containing oxidases underlies light-induced production of H2O2 in mammalian cells. Proc Natl Acad Sci USA 96, 6255-60
Jacobson K, Rajfur Z, Vitriol E, Hahn K (2008) Chromophore-assisted laser inactivation in cell biology. Trends Cell Biol 18, 434-50
Kanacher T, Schultz A, Linder J U, Schultz J E (2002) A GAF-domain-regulated adenylyl cyclase from Anabaena is a self-activating cAMP switch. EMBO J 21, 3672-80
Kehoe D M, Li L, Alvey R M. Apr. 15, 2010. US Patent Publication No. US 2010/0093051 for “Light Regulated Transcription System For Use In Prokaryotic Organisms”.
Kuzin A, Chen Y, Seetharaman J, Mao M, Xiao R, Ciccosanti C, Foote E L, Wang H, Everett J K, Nair R, Acton T B, Rost B, Montelione G T, Tong L, Hunt J F (2009) X-Ray Structure of Protein (EALIGGDEF domain protein) from M. capsulatus, Northeast Structural Genomics Consortium Target McR174C. PDB 3ICL.
Landry Y, Niederhoffer N, Sick E, Gies J P (2006) Heptahelical and other G-protein-coupled receptors (GPCRs) signaling. Curr Med Chem. 13, 51-63
Lehninger, A L, Nelson D L, Cox M M. Principles of Biochemistry, 2nd Ed.; Worth Publishers, Inc.: N.Y., 2000
Leung D W, Otomo C, Chory J, Rosen M K (2008) Genetically encoded photoswitching of actin assembly through the Cdc42-WASP-Arp2/3 complex pathway. Proc Natl Acad Sci USA 105, 12797-802
Levskaya A, Weiner O D, Lim W A, Voigt C A (2009) Spatiotemporal control of cell signaling using a light-switchable protein interaction. Nature 461, 997-1001
Levskaya A. Chevalier A A, Tabor J J, Simpson Z B, Lavery L A, Levy M, Davidson E A, Scouras A, Ellington A D, Marcotte E M, Voigt C A (2005) Synthetic biology: engineering Escherichia coli to see light. Nature 438, 441-2
Li P, Gao X-G, Arellano R O, Renugopalakrishnan V (2001) Glycosylated and Phosphorylated Proteins—Expression in Yeast and Oocytes of Xenopus: Prospects and Challenges—Relevance to Expression of Thermostable Proteins. Prot Expres Purif 22, 369-80
Linder J U (2006) Class III adenylyl cyclases: molecular mechanisms of catalysis and regulation. Cell Mol Life Sci 63, 1736-51
Liu X, Tonegawa S (2010) Optogenetics 3.0. Cell 141, 22-24
Mackenzie S H, Clay C (2008) Targeting cell death in tumors by activating caspases. Curr Cancer Drg Targets 8, 190-209
Maheshwari S C, Khurana J P, Sopory S K (1999) Novel light-activated protein kinases as key regulators of plant growth and development. J Biosci 24, 499-514
Miesenböck G (2009) The optogenetic catechism. Science 326, 395-9
Mills E, Chen X, Pham E, Wong S S, Truong K (2012) Engineering a photoactivated caspase-7 for rapid induction of apoptosis. ACS Synth Blol 3, 75-82
Möglich A, Yang X, Ayers R A, Moffat K (2010) Structure and function of plant photoreceptors. Annu Rev Plant Biol 61, 21-47
Möglich A, Ayers R A, Moffat K (2009) Design and signaling mechanism of light-regulated histidine kinases. J Mol Biol 385, 1433-44
Möglich A, Moffat K (2010) Engineered photoreceptors as novel optogenetic tools. Photochem Photobiol Sci 9, 1286-1300
Müller K, Weber W (2013) Optogenetic tools for mammalian systems. Mol BioSyst 9, 596-608
Pathak G P, Vrana J D, Tucker C L (2013) Optogenetic control of cell function using engineered photoreceptors. Biol Cell 105, 59-72
Pei J, Grishin N V (2001) GGDEF domain is homologous to adenylyl cyclase. Proteins 42, 210-6
Punta M, Coggill P C, Eberhardt R Y, Mistry J, Tate J, Boursnell C, Pang N, Forslund K, Ceric G, Clements J, Heger A, Holm L, Sonnhammer E L, Eddy S R, Bateman A, Finn R D (2012) The Pfam protein families database. Nucl Acids Res 40 (Database issue), D290-301
Piatkevich K D, Subach F V, Verkhusha V V (2013) Engineering of bacterial phylochromes for near-infrared imaging, sensing, and light-control in mammals. Chem Soc Rev 42, 3441-52
Pop C, Feeney V, Tripathy A, Clark A C (2003) Mutations in the procaspase-3 dimer interface affect the activity of the zymogen. Biochemistry 42, 12311-20
Quail P H, Huq E, Tepperman J, Sato S (2005) U.S. Pat. No. 6,858,429 for Universal Light-Switchable Gene Promoter System.
Rockwell N C, Su Y S, Lagarias J C (2006) Phytochrome structure and signaling mechanisms Annu Rev Plant Biol 57, 837-58
Römling U, Galperin M Y, Gomelsky M (2013) Cyclic di-GMP: the first 25 years of a universal bacterial second messenger. Microbiol Mol Biol Rev 77, 1-52
Ryjenkov D A, Tarutina M, Moskvin O V, Gomelsky M (2005) Cyclic diguanylate is a ubiquitous signaling molecule in bacteria: Insights into the biochemistry of the GGDEF protein domain. J Bacteriol 187, 1792-8
Ryu M H, Moskvin O V, Siltberg-Liberles J, Gomelsky M (2010) Natural and engineered photoactivated nucleotidyl cyclases for optogenetic applications. J Biol Chem 285, 41501-8
Ryu M H, Gomelsky M (2014) Near-infrared light responsive synthetic c-di-GMP module for optogenetic applications. ACS Synth Biol. January 28. [Epub ahead of print] DOI: 10.1021/sb400182x
Ryu M H, Kang I H, Nelson M D, Jensen T M, Lyuksyutova Al, Siltberg-Liberles J, Raizen D M, Gomelsky M. 2014. Engineering adenylate cyclases regulated by near-infrared window light. Proc Natl Acad Sci USA June 30. pii: 201324301. [Epub ahead of print].
Sambrook J, Fritsch E F, Maniatis T (1989). Molecular Cloning: a Laboratory Manual, 2nd ed. Cold Spring Harbor, N.Y.: Cold Spring Harbor Laboratory
Schade M A, Reynolds N K, Dollins C M, Miller K G (2005) Mutations that rescue the paralysis of Caenorhabditis elegans ric-8 (synembryn) mutants activate the G alpha(s) pathway and define a third major branch of the synaptic signaling network. Genetics 169, 631-49
Schirmer T, Jenal U (2009) Structural and mechanistic determinants of c-di-GMP signalling. Nat Rev Microbiol 7, 724-35
Schröder-Lang S, Schwärzel M, Seifert R. StrOnker T, Kateriya S, Looser J, Watanabe M, Kaupp U B, Hegemann P, Nagel G (2007) Fast manipulation of cellular cAMP level by light in vivo. Nat Methods 4, 39-42
Shu X, Royant A, Lin M Z, Aguilera T A, Lev-Ram Varda, Steinbach P A, Tsien R Y (2009) Mammalian expression of infrared fluorescent proteins engineered from a bacterial phytochrome. Science 324, 804-7
Sinha S C, Sprang S R (2006) Structures, mechanism, regulation and evolution of class III nucleotidyl cyclases. Rev Physiol Biochem Pharmacol 157, 105-40
Sjulson L, Miesenbdck G (2008) Photocontrol of neural activity: Biophysical mechanisms and performance in vivo. Chem Rev 108, 1588-1602
Sohal V S, Zhang F, Yizhar O, Deisseroth K (2009) Parvalbumin neurons and gamma rhythms enhance cortical circuit performance. Nature 459, 698-702
Sorokina O, Kapus A, Terecske K, Dixon L E, Kozma-Bognar L, Nagy F, Millar A J (2009) A switchable light-input, light-output system modelled and constructed in yeast. J Biol Eng 3, 15
Stierl M, Stumpf P, Udwari D, Gueta R, Hagedorn R, Losi A, Gärtner W, Petereit L, Efetova M, Schwarzel M, Oertner T G, Nagel G, Hegemann P (2011) Light modulation of cellular cAMP by a small bacterial photoactivated adenylyl cyclase, bPAC, of the soil bacterium Beggiatoa. J Biol Chem 286, 1181-8
Stinchcomb D T, Shaw J E, Carr S H, Hirsh D (1985) Extrachromosomal DNA transformation of Caenorhabditis elegans. Mol Cell Biol 5, 3484-96
Strickland D, Moffat K, Sosnick T R (2008) Light-activated DNA binding in a designed allosteric protein. Proc Natl Acad Sci USA 105, 10709-14
Takala H, Björling A, Berntsson O, Lehtivuori H, Niebling S, Hoernke M, Kosheleva I, Henning R, Menzel A, Ihalainen J A, Westenhoff S (2014) Signal amplification and transduction in phytochrome photosensors. Nature 509, 245-8
Tarutina M, Ryjenkov D A, Gomelsky M (2006) An unorthodox bacteriophytochrome from Rhodobacter sphaeroides involved in turnover of the second messenger c-di-GMP. J Biol Chem 281, 34751-8
Toettcher J E, Voigt C A, Weiner O D, Lim W A (2011) The promise of optogenetics in cell biology: interrogating molecular circuits in space and time. Nature Meth 8, 35-38
Tønnesen J, Sørensen A T, Deisseroth K, Lundberg C, Kokaia M (2009) Optogenetic control of epileptiform activity. Proc Natl Acad Sci USA 106, 12162-7
Topal H, Fulcher N B, Bitterman J, Salazar E, Buck J, Levin L R, Cann M J, Wolfgang M C, Steegborn C (2012) Crystal structure and regulation mechanisms of the CyaB adenylyl cyclase from the human pathogen Pseudomonas aeruginosa. J Mol Biol 416, 271-86
Tsai H C, Zhang F, Adamantidis A, Stuber G D, Bonci A, de Lecea L, Deisseroth K (2009) Phasic firing in dopaminergic neurons is sufficient for behavioral conditioning. Science 324, 1080-4
Tyszkiewicz A B, Muir T W (2008) Activation of protein splicing with light in yeast. Nature Meth 5, 303-5
Ulijasz A T, Vierstra R D (2011) Phytochrome structure and photochemistry: recent advances toward a complete molecular picture. Curr Opin Plant Biol 14, 498-506
Walters J, Pop C, Scott F L, Drag M, Swartz P, Mattos C, Salvesen G S, Clark A C (2009) A constitutively active and uninhibitable caspase-3 zymogen efficiently induces apoptosis. Biochem J 424, 335-45.
Wan S, Parrish J A, Anderson R R, Madden M (1981) Transmittance of nonionizing radiation in human tissues. Photochem Photobiol 34, 679-81
Weissenberger S, Schultheis C, Liewald J F, Erbguth K, Nagel G, Gottschalk A (2011) PACalpha—an optogenetic tool for in vivo manipulation of cellular cAMP levels, neurotransmitter release, and behavior in Caenorhabditis elegans. J Neurochem 116, 616-25.
Weissleder R (2001) A clearer vision for in vivo imaging. Nature Biotechnol 19, 316-7
Wu Y I, Frey D Lungu O., Jaehrig A, Schlichting I, Kuhlman B, Hahn K M (2009) A genetically encoded photoactivatable Rac controls the motility of living cells. Nature 461, 104-8
Yang X, Kuk J, Moffat K (2008) Crystal structure of Pseudomonas aeruginosa bacteriophytochrome: photoconversion and signal transduction. Proc Natl Acad Sci USA 105, 14715-20
Yang X, Kuk J, Moffat K (2009) Conformational differences between the Pfr and Pr states in Pseudomonas aeruginosa bacteriophytochrome. Proc Natl Acad Sci USA 106, 15639-44
Yazawa M, Sadaghiani A M, Hsueh B, Dolmetsch R E (2009) Induction of protein-protein interactions in live cells using light. Nat Biotechnol 27, 941-5
Vera, Aris A, Daura X, Martinez M A, Villaverde A (2005) Engineering the E. coli beta-galactosidase for the screening of antiviral protease inhibitors. Biochem Biophys Res Commun 329, 453-6
Vuillet L, Kojadinovic M, Zappa S, Jaubert M, Adriano J. M., Fardoux J I, Hannibal L, Pignol D, Vermeglio A, Giraud E (2007) Evolution of a bacteriophytochrome from light to redox sensor. EMBO J 26, 3322-31
Zähringer F. Lacanna E, Jenal U, Schirmer T, Boehm A (2013) Structure and signaling mechanism of a zinc-sensory diguanylate cyclase. Structure 21, 1149-57
Zhang J, Stankey, R J, Vierstra R D (2013) Structure-guided engineering of plant phytochrome B with altered photochemistry and light signaling. Plant Physiol 161, 1445-57
Zimmer M (2009) GFP: from jellyfish to the Nobel prize and beyond. Chem Soc Rev 38, 2823-32

1. A homodimeric fusion protein controllable by far red and/or near-infrared (NIR) light, said fusion protein comprising a photoreceptor module comprising:
a. a bacteriophytochrome; and
b. a heterologous output module capable of producing a desired activity; wherein said homodimeric fusion protein comprises two monomers that each comprise:
(1) a photoreceptor module of a bacteriophytochrome; and
(2) a heterologous output module capable of being activated upon homodimerization to perform said desired activity;
wherein said monomers are not active when separated, but are capable of combining to form homodimers that are controllable by far red or NIR light.

2. The homodimeric fusion protein of claim 1 also comprising a linker sequence between said photoreceptor module and said output module.

3. The homodimeric fusion protein of claim 1 made by a method comprising:
a. identifying candidate output domains based on 3D structures or models;
b. identifying candidate protein fusion sites; and
c. estimating lengths of α-helices linking said output modules to said photoreceptor modules;
d. producing a plurality of DNA molecules, each encoding a said monomer of a said homodimeric fusion protein that has at least one unique fusion site;
e. screening said DNA molecules for their ability to produce homodimeric photoactive fusion proteins capable of performing said desired activity by a method comprising:
i. transforming a non-human test organism with a plurality of different said DNA molecules such that a different said fusion protein is expressed in each test organism;
ii. allowing the expressed fusion proteins to bind bacteriophytochrome chromophore and form homodimeric proteins; and
iii. applying selected wavelengths of NIR light to said transformed organisms and determining the level of said desired activity of said fusion proteins in said organisms in the presence and absence of said selected wavelengths of light;
wherein the level of said desired activity of said fusion proteins is controllable by NIR light when the level of said desired activity is changed by the presence and/or absence of NIR light having said selected wavelengths.

4. The homodimeric fusion protein of claim 3 wherein said process also comprises designing additional fusion sites and linkers for said fusion proteins and producing DNA encoding the additional DNA molecules encoding fusion proteins comprising said additional fusion sites and linkers, transforming suitable organisms with this DNA, expressing the DNA, and screening the resultant fusion proteins for additional fusion proteins controllable by NIR light.

5. A set of homodimeric fusion proteins of claim 2 wherein said linkers differ in length by the length of one or more helical turns to produce additional candidate fusion proteins.

6. The homodimeric fusion protein of claim 1 which has a high ratio of activity in the light versus dark or vice versa.

7. The homodimeric fusion protein of claim 1 which is selected from the group consisting of light-activated nucleotidyl cyclases, light-activated uncleavable procaspase-3, protein kinases, proteases, and DNA-binding and RNA-binding proteins.

8. The homodimeric fusion protein of claim 7 which is a light-activated adenylyl cyclase or a light-activated guanidyl cyclase.

9. The homodimeric fusion protein of claim 7 in which protein inactivity is induced by light, said protein comprising a sequence selected from the group consisting of SEQ ID NO:1, SEQ ID NO:19, and SEQ ID NO:20.

10. The homodimeric fusion protein of claim 7 in which protein activity is induced by light, said protein comprising a sequence selected from the group consisting of SEQ ID NO: 2, SEQ ID NO:6, SEQ ID NO:9, SEQ ID NO:10, SEQ ID SEQ ID NO:21, SEQ ID NO:25, and SEQ ID NO:28, SEQ ID NO:29.

11. The homodimeric fusion protein of claim 1 wherein said bacteriophytochrome photoreceptor module is from the BphG1 protein from Rhodobacter sphaeroides.

12. A recombinant DNA molecule encoding the homodimeric fusion protein of claim 1.

13. A host organism capable of expressing the fusion protein of claim 1 which is transformed with the DNA sequence of claim 12.

14. The host organism of claim 13 which is a cultured organism selected from the group consisting of bacteria, yeast, plant, insect or mammalian cells selected or modified so as to detectably exhibit the level of activity of said expressed fusion protein controllable by the presence or absence of far red or NIR light.

15. The host organism of claim 13 which is a multicellular organism selected from the group consisting of insects, plants, and animals.

16. The host organism of claim 13 which is a human.

17. The host organism of claim 13 also comprising heme oxygenase introduced into the organism from outside or by transforming the organism with a heme oxygenase gene or heme oxygenase precursor gene.

18. A method for controlling an in vivo process in a host which is a living cell or organism comprising:
a. introducing into the cell or organism, or selected portion of the organism a DNA sequence of claim 12 encoding a homodimeric fusion protein comprising a photoreceptor module comprising a bacteriophytochrome and a heterologous output module capable of modulating said process;
b. allowing said fusion protein to be expressed in said host; and
c. applying NIR light of a selected wavelength to the host or preventing NIR light of a selected wavelength from contacting the host; thereby modulating the process under control of NIR light.

19. The method of claim 18 wherein said process is selected from the group consisting of metabolic processes, signal transduction, cell apoptosis, cell proliferation, cell adhesion, and cell differentiation.

20. The method of claim 19 wherein said process is selected from the group consisting of cyclic AMP production; muscle activity, and heart rate, and hormone production.


SEQUENCE LISTING (34 sequences)

SEQ ID NO: 1 - protein, 22 aa, Rhodobacter sphaeroides
MAQRTRAELARLRHYDERKEVT

SEQ ID NO: 2 - protein, 19 aa, Rhodobacter sphaeroides
MAQRTRAELARLRERKEVT

SEQ ID NO: 3 - protein, 18 aa, Rhodobacter sphaeroides
MAQRTRAELARLERKEVT

SEQ ID NO: 4 - protein, 17 aa, Rhodobacter sphaeroides
MAQRTRAELARERKEVT

SEQ ID NO: 5 - protein, 16 aa, Burkholderia sp.
MAQRTRAELAERKEVT

SEQ ID NO: 6 - protein, 15 aa, Prauserella rugosa
MAQRTRAELERKEVT

SEQ ID NO: 7 - protein, 14 aa, Artificial Sequence
  Engineered peptide
MAQRTRAEERKEVT

SEQ ID NO: 8 - protein, 13 aa, Artificial Sequence
  Engineered peptide
MAQRTRAERKEVT

SEQ ID NO: 9 - protein, 12 aa, Artificial Sequence
  Engineered peptide
MAQRTRERKEVT

SEQ ID NO: 10 - protein, 11 aa, Artificial Sequence
  Engineered peptide
MAQRTERKEVT

SEQ ID NO: 11 - protein, 10 aa, Artificial Sequence
  Engineered peptide
MAQRERKEVT

SEQ ID NO: 12 - protein, 9 aa, Artificial Sequence
  Engineered peptide
MAQERKEVT

SEQ ID NO: 13 - protein, 29 aa, Rhodobacter sphaeroides
IAAEMAQRTRAELARLRHYDMVGERKEVT

SEQ ID NO: 14 - protein, 43 aa, Artificial Sequence
  Chimeric peptide
IAAEMAQRTRAELARLRHYDPLTGILANLGEDALMVGERKEVT

SEQ ID NO: 15 - protein, 42 aa, Artificial Sequence
  Chimeric peptide
IAAEMAQRTRAELARLRHYDPLTGILANGEDALMVGERKEVT

SEQ ID NO: 16 - protein, 41 aa, Artificial Sequence
  Chimeric peptide
IAAEMAQRTRAELARLRHYDPLTGILANEDALMVGERKEVT

SEQ ID NO: 17 - protein, 39 aa, Artificial Sequence
  Chimeric peptide
IAAEMAQRTRAELARLRHYDPLTGILANALMVGERKEVT

SEQ ID NO: 18 - protein, 34 aa, Artificial Sequence
  Chimeric peptide
IAAEMAQRTRAELARLRHYDPLTGILANERKEVT

SEQ ID NO: 19 - protein, 29 aa, Rhodobacter sphaeroides
IAAEMAQRTRAELARLRHYDMVGERKEVT

SEQ ID NO: 20 - protein, 26 aa, Rhodobacter sphaeroides
IAAEMAQRTRAELARLRHYDERKEVT

SEQ ID NO: 21 - protein, 23 aa, Rhodobacter sphaeroides
IAAEMAQRTRAELARLRERKEVT

SEQ ID NO: 22 - protein, 22 aa, Rhodobacter sphaeroides
IAAEMAQRTRAELARLERKEVT

SEQ ID NO: 23 - protein, 21 aa, Rhodobacter sphaeroides]
IAAEMAQRTRAELARERKEVT

SEQ ID NO: 24 - protein, 20 aa, Rhodobacter sphaeroides
IAAEMAQRTRAELAERKEVT

SEQ ID NO: 25 - protein, 19 aa, Artificial Sequence
  Engineered peptide
IAAEMAQRTRAELERKEVT

SEQ ID NO: 26 - protein, 18 aa, Rhodobacter sphaeroides
IAAEMAQRTRAEERKEVT

SEQ ID NO: 27 - protein, 17 aa, Rhodobacter sphaeroides
IAAEMAQRTRAERKEVT

SEQ ID NO: 28 - protein, 16 aa, Rhodobacter sphaeroides
IAAEMAQRTRERKEVT

SEQ ID NO: 29 - protein, 15 aa, Artificial Sequence
  Engineered peptide
IAAEMAQRTERKEVT

SEQ ID NO: 30 - protein, 14 aa, Artificial Sequence
  Engineered peptide
IAAEMAQRERKEVT

SEQ ID NO: 31 - protein, 13 aa, Artificial Sequence
  Engineered peptide
IAAEMAQERKEVT

SEQ ID NO: 32 - protein, 12 aa, Artificial Sequence
  Engineered peptide
IAAEMAERKEVT

SEQ ID NO: 33 - protein, 11 aa, Artificial Sequence
  Engineered peptide
IAAEMERKEVT

SEQ ID NO: 34 - protein, 779 aa, Artificial Sequence
  Engineered protein
MARGCLMTISGGTFDPSICEMEPIATPGAIQPHGALMTARADSGRVAHASVNLGEILGLPAASVLGAPIGEVIGRVNEILLREARRSGSETPETIGSFRRSDGQLLHLHAFQSGDYMCLDIEPVRDEDGRLPPGARQSVIETFSSAMTQVELCELAVHGLQLVMGYDRVMAYRFGADGHGEVIAERRRQDLEPYLGLHYPASDIPQIARALYLRQRVGAIADACYRPVPLLGHPELDDGKPLDLTHSSLRSVSPVHLDYMQNMNTAASLTIGLADGDRLWGMLVCHNTTPRIAGPEWRAAAGMIGQVVSLLLSRLGEVENAAETLARQSTLSTLVERLSTGDTLAAAFVAADQLILDLVGASAAVVRLAGHELHFGRTPPVDAMQKVLDSLGRPSPLEVLSLDDVTLRHPELPELLAAGSGILLLPLTSGDGDLIAWFRPEHVQTITWGGNPAEHGTWNPATQRMRPRASFDAWKETVTGRSLPWTSAERNCARELGEAIAAEMAQRTWERKEVTVLFSDIRGYTTLTENLGAAEVVSLLNQYFETMVEAVFNYEGTLDKFIGDALMAVFGAPLPLTENHAWQAVRSALDMRQRLKEFNQRRIIQAQPQIKIGIGISSGEVVSGNIGSHKRMDYTVIGDGVNLSSRLETVTKEYGCDIILSEFTYQLCSDRIRVRQLDKIRVKGKHQAVNIYELISDRSTPLDDNTQEFLFHYHNGRTAYLVRDFTQAIACFNSAKHIRPTDQAVNIHLERAYNYQQTPPPPQWDGVWTIFTKHHHHHH